(19) United States
(12) Patent Application Publication
(10) Pub. No.: US 2011/0195111 A1
(43) Pub. Date: Aug. 11, 2011

(54) AQUEOUS COMPOSITIONS AND METHODS

(75) Inventors: B. Michael Butters, Lacey, WA (US); John T. Butters, Del Mar, CA (US); Christine Bonzon, San Diego, CA (US); Marco Gymnopoulos, San Diego, CA (US); Mayra Montes Camacho, National City, CA (US)

(73) Assignee: NATIVIS, INC., La Jolla, CA (US)

(21) Appl. No.: 12/972,089

(22) Filed: Dec. 17, 2010

Related U.S. Application Data
(60) Provisional application No. 61/287,559, filed on Dec. 17, 2009.

(51) Int. Cl. A61K 31/337; A61K 9/127; A61P 35/00; A61K 31/7105; C12N 5/09; G01J 3/44; G01J 3/00; G01R 33/12; B82Y 5/00

(52) U.S. Cl. 424/450; 514/449; 514/44 A; 435/375; 356/301; 356/51; 324/228; 977/773

(57) ABSTRACT

A method of forming an aqueous composition effective to produce an agent-specific effect on an agent-responsive chemical or biological system, when the composition is added to the system, is disclosed. The composition is formed by exposing an aqueous medium to a low-frequency, time-domain signal derived from the agent, until the aqueous medium acquires a detectable agent activity. Exemplary compositions are formed by exposure to a paclitaxel signal or a signal derived from a therapeutic oligonucleotide, such as GAPDH antisense RNA and PCSK9 antisense RNA. Also disclosed are methods for confirming the activity of the composition, and for preparing and testing the activity of the compositions.

This application claims the benefit of priority to U.S. provisional patent application No. 61/287,559 filed on Dec. 17, 2009, which is incorporated in its entirety herein by reference.

### FIELD OF THE INVENTION

The present invention relates to an aqueous composition effective to mimic the effect of an agent on a chemical, biochemical, or biological system, and to methods and systems for making, using and testing the composition.

### BACKGROUND OF THE INVENTION

One of the accepted paradigms in the fields of chemistry and biochemistry is that chemical or biochemical effector agents, e.g., molecules, interact with target biological systems through various physicochemical forces, such as ionic, charge, or dispersion forces or through the cleavage or formation of covalent or charge-induced bonds. These forces presumably involve field effects, e.g., electrostatic and magnetic field effects, by which the presence of the effector influences the condition or response of the target.

One question raised by this paradigm is whether interactions between effector and target require the presence of the effector itself or whether at least some critical effector-target interactions can be achieved by simulating field effects associated with effector molecules with signals derived from the effector molecules. Studies undertaken to examine the interaction between effector-molecule signals and biological targets were reported in co-owned PCT applications WO 2006/073491 A2 and WO 2008/063654 A2, both of which are incorporated by reference herein. These applications describe studies in which low-frequency time-domain signals recorded for a number of bio-active compounds (effectors), in accordance with apparatus and methods detailed in the applications, were used in induce compound-specific effects in biological target systems.

PCT application WO 2006/073491, published Jul. 13, 2006 discloses studies in which (a) low-frequency time-domain signals recorded for L(+) arabinose were shown to induce the araC-PBAD bacterial operon, as discussed on pages 47-50 of the application, with respect to FIGS. 30C-30F; (b) low-frequency signals recorded for glyphosphate, the active ingredient in a well-known herbicide, were shown to substantially inhibit stem growth in pea sprouts, as discussed on pages 50-51 of the application, with respect to FIGS. 31 and 32A and 32B; (c) low-frequency signals recorded for gibberelic acid, a plant hormone, were shown to significantly increase average stem length in live sugar pea sprouts, as discussed on pages 51-53 of the application, with respect to FIG. 33; and (d) low-frequency signals recorded for phepropeptin, a proteasome inhibitor, were shown to decrease the activity of the 20S proteosome enzyme, as discussed on pages 53-54 of the application, with respect to FIG. 34.

WO 20081063654 A2, published May 9, 2008, details studies in which low-frequency time-domain signals for the anti-tumor compound paclitaxel, generated in accordance with methods disclosed herein, were shown to be effective in reducing tumor growth in animals injected with glioblastoma cells, when the animals were exposed to an electromagnetic field generated by the signal over a several-week period.

Among the findings from the studies described above is that the ability of agent-specific, time-domain signals to transduce (affect) a biochemical or biological target system can be optimized by a number of strategies. One of these strategies involves scoring recorded time-domain signals by one or more scoring algorithms to identify those signals that contain the highest spectral information. This scoring is used to screen recorded time-domain signals for those that are most likely to give a strong transduction effect. An improvement in this strategy is to record time-domain signals at each of a number of different magnetic-signal injection conditions, by injecting different levels of white noise or DC offset during recording, and scoring the resulting signals for highest spectral information. These strategies are detailed in both of the above-cited PCT applications.

A third strategy, disclosed in the '654 application, is designed particularly for applications in which a recorded time-domain signal is intended for transducing an animal system, for example, for treating a disease condition in a subject. The strategy involves screening time-domain signals for their ability to effectively transduce an in vitro target system that includes at least some of the critical biological response components of the animal system. The strategy has the advantage that a large number of candidate signals can be easily screened for actual transduction effect, to identify optimal transducing signals. The strategy is preferably combined with one or both of the above signal-scoring methods, using the highest-scoring signals as candidates for the in vitro transduction screening.

Independently, a number of scientific groups have reported on the structure and stability of clustered water in pure and solute-containing water samples, including structured water formed at interfaces. See, for example, studies cited in the websites of Dr. Rustum Roy, late of the Pennsylvania State University (rustumroy.com); Dr. Gerald Pollack at the University of Washington (www.depts.washington.eduibioe/people/core/pollack.html)); Dr. Martin Chaplin of the London South Bank University (1.lsbu.ac.uk/wate); and Dr. Emilio Del Guidice (isi.it/progetti/workshop-complexity09/pres_DelGiudice.pdf). Among the findings of these groups is that water interacts with electromagnetic radiation to form stable macroscopic structures that can be detected by a number of physical and spectroscopic tools; (See, for example, del Guidice, E., et al., Physical Review, 74:022105-1 (2006); Pollack, G., uwtv.org/programs/displayevent.aspx?rID=22222): Chai, B. et al, J. Phys. Chem. B, 2009, 113:13953-13958; Rao, M. L., et al., Current Science Research Communications, 98(1); 1500, Jun., 2010.

### SUMMARY OF THE INVENTION

In one aspect, the invention includes an aqueous anti-tumor composition produced by treating an aqueous medium free of paclitaxel, a paclitaxel analog, or other cancer-cell inhibitory compound with a low-frequency, time-domain signal derived from paclitaxel or an analog thereof, until the aqueous medium acquires a detectable paclitaxel activity, as evidenced by the ability of the composition to (i) inhibit growth of human glioblastoma cells when the composition is added to the cells in culture, over a 24 hour culture period, under standard culture conditions, and (ii), to inhibit growth of a paclitaxel responsive tumor when administered to a subject having such a tumor.

The aqueous medium in the composition may be mechanically disrupted, an interfacial aqueous medium containing gas bubbles, or a mechanically disrupted interfacial aqueous medium containing gas bubbles.

The composition may have an activity, expressed in terms of paclitaxel concentration, of between 1 and 100 uM, The anti-tumor activity of the composition may be abolished by treatments that disrupt signal-related water structures, such as heating the composition to a temperature of 70° C. or greater, or by cooling the composition to below freezing. The composition may contain between 0.5 to 10% ethanol by volume.

Also disclosed is a method of forming the above composition, by the steps of:

(a) placing an aqueous medium within the sample region of an electromagnetic-coil device, and

(b) exposing the aqueous medium to a magnetic field generated by supplying to the device, a low-frequency, time domain signal derived from paclitaxel or an analog thereof, at a signal current calculated to produce a magnetic field strength in the range between 1 G (Gauss) and 10⁻⁸ G, for a period sufficient to render the aqueous medium effective in inhibiting the growth of tumor cells, e.g., glioblastoma cells, in culture, or inhibiting tumor growth in viva, e.g., implanted glioblastoma cells in an animal model.

The low-frequency, time domain signal used in step (b) of the method may be produced by the steps of

(i) placing in a sample container having both magnetic and electromagnetic shielding, an aqueous sample of paclitaxel or analog thereof, wherein the sample acts as a signal source for low-frequency molecular signals; and wherein the magnetic shielding is external to a cryogenic container;

(ii) recording one or more time-domain signals composed of sample source radiation in the cryogenic container, and

(iii) identifying from among the signals recorded in step (ii), a signal effective to mimic the effect of paclitaxel in a paclitaxel-responsive system, when the system is exposed to a magnetic field produced by supplying the signal to electromagnetic transducer coil(s) at a signal current calculated to produce a magnetic field strength in the range between 1 G to 10⁻⁸ G.

The concentration of the paclitaxel or analog thereof in the sample may be between 10⁻¹¹ to 10⁻¹⁹ M, and the sample may be treated, prior to being placed within the sample region of the device, to form one of: (i) a mechanically disrupted sample medium, (ii) an interfacial sample medium containing gas bubbles, (iii) a mechanically disrupted interfacial sample medium containing gas bubbles, or (iv) a suspension of liposomes or other nanoparticles.

The method may further include, before and/or after step (b), treating the aqueous medium to form one of: (i) a mechanically disrupted aqueous medium, (ii) an interfacial aqueous medium containing gas bubbles, (iii) a mechanically disrupted interfacial aqueous medium containing gas bubbles, or (iv) a suspension of liposomes or other nanoparticles.

Also disclosed are methods for confirming the cancer-cell inhibitory activity of the aqueous composition above. One exemplary method involves interrogating the composition by spectroscopic analysis capable of detecting water structures produced when the aqueous medium is exposed to the signal, and confirming that the spectral characteristics observed for the sample, e.g., spectral peak frequencies and amplitudes, are similar to those of a similarly-prepared aqueous composition. Methods that have been used in characterizing condensed or electromagnetic-field induced domains in water are (i) ultraviolet (UV) or ultraviolet-visible (UV-Vis) absorption spectroscopy (see, for example, Chai, B., et al, J. Phys Chem A, 2009, 112:2242-2247)), (ii) IR spectroscopy (e.g., Roy, R, Materials Res. Innov, 2005, 9(4):1433 and Rao, M., et al., Materials Letters, 2008, 62(10-11):1487-1490), including Fourier-transform infrared (FTIR) absorption spectroscopy (see, for example, Amrein, A., et al., J. Phys Chem, 1988 92(19): 5455-5466), and (iii) Raman spectroscopy (e.g., Roy, ibid). In an alternative approach, water structure in the aqueous medium may be analyzed by atomic force microscopy (AFM), and compared with AFM plots of aqueous compositions with known activity. Methods for analyzing water structure by AFM has been described, for example, in Michaelides, A. et al., Nature Mater. 6, 597 (2007) and Pan, a et al., Phys, Rev, Lett. 101, 155709 (2008).

In a more general aspect, the invention includes a method of forming an aqueous composition effective to produce an agent-specific effect on an agent-responsive chemical or biological system, when the composition is added to the system. The method includes the steps of:

(a) placing an aqueous medium within the sample region of an electromagnetic-coil device; and

(b) exposing the aqueous medium to a magnetic field generated by supplying to the device, a low-frequency, time-domain agent-specific signal, at a signal current calculated to produce a magnetic field strength in the range between 1 G (Gauss) and 10⁻⁸ G, for a period sufficient to render the aqueous medium effective to mimic one or more agent-specific effects on an agent-responsive system.

The low-frequency, time domain signal used in step (b) may be produced by the steps of:

(i) placing in a sample container having both magnetic and electromagnetic shielding, an aqueous sample of the agent, wherein the sample acts as a signal source for low-frequency molecular signals; and wherein the magnetic shielding is external to a cryogenic container;

(ii) recording one or more time-domain signals composed of sample source radiation in the cryogenic container, and

(iii) identifying from among the signals recorded in step (ii), a signal effective to mimic the effect of the agent in an agent-responsive system, when the system is exposed to a magnetic field produced by supplying the signal to electromagnetic transducer coil(s) at a signal current calculated to produce a magnetic field strength in the range between 1 G to 10⁻⁸ G.

The concentration of the agent in the sample may be between 10⁻¹⁰ to 10⁻¹⁶ μM, and the sample may be treated, prior to being placed within the sample region of the device, to form (i) a mechanically disrupted aqueous medium, (ii) an interfacial aqueous medium containing gas bubbles and (iii) a mechanically disrupted interfacial aqueous medium containing gas bubbles.

The method may include, before and/or after step (b), treating the aqueous medium to form one of: (i) a mechanically disrupted aqueous medium, (ii) an interfacial aqueous medium containing gas bubbles, (iii) a mechanically disrupted interfacial aqueous medium containing gas bubbles, or (iv) a suspension of liposomes or other nanoparticles. For example, the method of may include, before and/or after step (b) mechanically agitating the aqueous medium to form a mechanically disrupted aqueous medium.

The agent may be, for example, (i) paclitaxel, (ii) an analog of paclitaxel, or (iii) a therapeutic oligonucleotide, such as GAPDH antisense RNA or PCSK9 antisense RNA.

In a related aspect, the invention includes an aqueous composition produced by treating an aqueous medium free of oligonucleotide with a low-frequency, time-domain signal derived from a therapeutic oligonucleotide, until the aqueous medium acquires a detectable activity associated with the therapeutic oligonucleotide. The therapeutic oligonucleotide from which the treating signal is derived may be, for example, GAPDH antisense RNA or PCSK9 antisense RNA.

The aqueous medium in the composition may be a mechanically disrupted medium, an interfacial aqueous medium containing gas bubbles, or a mechanically disrupted interfacial aqueous medium containing gas bubbles.

The composition may contain between 0.5 to 10% ethanol by volume. The agent-specific activity of the composition may be abolished by (i) heating the composition to a temperature greater than 70° C. or by (ii) cooling the composition to below freezing.

Also disclosed is a method for confirming the agent-specific activity of the above composition by the steps of: (a) generating a spectrum of the composition by a spectroscopic analysis capable of detecting condensed structures in water, and determining that the generated spectrum is similar in its spectral composition to the spectrum of a similarly prepared aqueous composition having a known agent-specific effect. Methods that have been used in characterizing in detecting condensed domains in water are (i) ultraviolet and UV-Vis spectroscopy, (ii) IR spectroscopy, including FTIR spectroscopy, and (iii) Raman spectroscopy, all as referenced above.

Further disclosed is a system for producing an aqueous composition intended to produce an agent-specific pharmaceutical effect on a mammalian subject, when the composition is administered in a pharmaceutically effective amount to the subject. The system includes (a) a coil device for treating an aqueous medium with a low-frequency, time-domain, agent-specific signal under conditions effective to convert the aqueous medium to an aqueous composition having agent-specific properties; and (b) a spectroscopic instrument for generating a spectrum of the composition, by which the spectral characteristics of the aqueous composition can be compared with those of an aqueous medium having a known activity. Suitable spectroscopic instruments include (i) a UV or UV-Vis spectrometer; (ii) an IR spectrometer, preferably with Fourier transform enhancement capabilities, and (iii) a Raman spectrometer.

In one embodiment, device (a) includes (i) a source of an agent-specific time-domain signal; (ii) an electromagnetic transduction coil device for receiving a vessel containing an aqueous medium within a vessel holder in the device, and (iii) an electronic interface between said source and said device, for supplying to the device, a source-signal current calculated to produce at an aqueous medium contained in a vessel at the sample region of the device, a magnetic field having a field strength in the range between 1 G to 10⁻⁸ G, over a time period sufficient to transform aqueous medium in said into said agent-specific composition.

In another embodiment, device (a) includes (i) an electromagnetic coil defining therewithin, a signal-transfer environment in which a first vessel containing a solution or suspension of the agent can be placed adjacent a second vessel containing an untreated aqueous medium, and (ii) means for supplying to the coil, an electric current having an oscillation frequency of 7.83 Hz, wherein supplying such current to the coil, with the two vessels in close proximity within the coil environment, over a given time period, e.g. 18-24 hours, is effective to transform the aqueous medium in the second vessel to one effective to produce an agent-specific effect on an agent-responsive chemical or biological system.

The system may further include a device for treating the aqueous medium to produce one of: (i) a mechanically disrupted aqueous medium, such as a vortexing device, (ii) an interfacial aqueous medium containing gas bubbles and (iii) a mechanically disrupted interfacial aqueous medium containing gas bubbles.

These and other objects and features of the invention will be more fully understood when the following detailed description of the invention is read in conjunction with the accompanying drawings.

### BRIEF DESCRIPTION OF THE DRAWINGS

FIG. 1 is a diagram of a signal-recording apparatus used in producing agent-specific, time-domain signals employed in the invention;

FIG. 2 is a diagram showing components of the signal-recording apparatus of FIG. 1;

FIG. 3 is a flow diagram of the signal recording and processing performed in producing an agent-specific time-domain signal employed in the invention;

FIG. 4 shows a high-level flow diagram of data flow for processing agent-specific time-domain signals employed in the invention;

FIG. 5 is a flow diagram of a histogram-bin algorithm used in scoring agent-specific time-domain signals employed in the invention;

FIG. 6 is a flow diagram of a power spectral density algorithm in accordance with another algorithm that can be used in scoring agent-specific time-domain signals employed in the invention;

FIG. 7 illustrates a transduction/exposure apparatus for applying a time-domain signal to an aqueous sample, and for recording spectrophotometrically, changes in the sample over time or at a selected end point;

FIGS. 8A-8C illustrates a general transduction/exposure system used in producing the composition of the invention (8A), a circuit diagram for an attenuator used in the system (8B), and operational features of the system (8C);

FIGS. 9A-9C show frequency-domain spectra of two paclitaxel signals with noise removed by Fourier subtraction (FIGS. 9A and 9B), and a cross-correlation of the two signals (FIG. 9C), showing agent-specific spectral features over a portion of the frequency spectrum;

FIG. 10 is a bar graph showing the viability of U87 glioma cells in culture after 24 hours in a culture medium previously exposed to a paclitaxel signal;

FIG. 11 plots the effect on U87 MG cell tumor growth in animals over a 26-day treatment period for: no treatment (X's, light line), white noise (X's, heavy line); treatment with paclitaxel vehicle alone (triangles, light line), treatment with paclitaxel (triangles, dark line); and treatment with water exposed to taxane signal (squares);

FIGS. 12A-12D are bar graphs showing changes in lipid profiles after oral administration of an aqueous composition formed by exposure to a signal from antisense to pCSK9;

FIG. 13 shows in schematic view a system for producing and testing an aqueous composition in accordance with an aspect of the invention; and

FIG. 14 is a flowchart of steps used in confirming an activity of an aqueous composition formed in accordance with the invention.

### DETAILED DESCRIPTION OF THE INVENTION

### I. Definitions

The terms below have the following meaning unless indicated otherwise.

“Electromagnetic shielding” refers to, e.g., standard Faraday electromagnetic shielding, or other methods to reduce passage of electromagnetic radiation.

“Time-domain signal” or “time-series signal” refers to a signal with transient signal properties that change over time.

“Low-frequency” refers to a frequency range from DC to about 50 kHz. A low-frequency time domain signal is one having its major frequency components in the 0-50 kHz range, typically 0-20 kHz range.

“Sample-source radiation” refers to magnetic flux or electromagnetic flux emissions resulting from molecular motion of a sample, or electromagnetic fields produced by short-range or long-range interactions between two of more molecules undergoing molecular motion. When sample source radiation is produced in the presence of an injected magnetic-field stimulus,” it is also referred to as “sample source radiation superimposed on injected magnetic field stimulus.”

“Stimulus magnetic field” or “magnetic-field stimulus” refers to a magnetic field produced by injecting (applying) to magnetic coils surrounding a sample, one of a number of electromagnetic signals that may include (i) white noise, injected at voltage level calculated to produce a selected magnetic field at the sample of between 0 and 1 G (Gauss), (ii) a DC offset, injected at voltage level calculated to produce a selected magnetic field at the sample of between 0 and 1 G, and (iii) a combination of (i) and (ii). The injected noise and/or offset may be varied incrementally and systematically, for generating a plurality of time-domain signals at different magnetic-filed conditions.

The “magnetic field strength” produced at the sample, by supplying a time domain signal to transduction coils, may be readily calculated using known electromagnetic relationships, knowing the shape and number of windings in the injection coil, the current applied to coils, and the distance between the injection coils and the sample, according to known methods as described below.

A “selected stimulus magnetic-field condition” refers to a selected voltage applied to a white noise or DC offset signal, or a selected sweep range, sweep frequency and voltage of an applied sweep stimulus magnetic field.

“White noise” means random noise or a signal having simultaneous multiple frequencies, e.g. white random noise or deterministic noise. “Gaussian white noise” means white noise having a Gaussian power distribution. “Stationary Gaussian white noise” means random Gaussian white noise that has no predictable future components. “Structured noise” is white noise that may contain a logarithmic characteristic which shifts energy from one region of the spectrum to another, or it may be designed to provide a random time element while the amplitude remains constant. These two represent pink and uniform noise, as compared to truly random noise which has no predictable future component. “Uniform noise” means white noise having a rectangular distribution rather than a Gaussian distribution.

“Frequency-domain spectrum” refers to a Fourier frequency plot of a time-domain signal.

“Spectral components” refer to singular or repeating qualities within a time-domain signal that can be measured in the frequency, amplitude, and/or phase domains. Spectral components will typically refer to signals present in the frequency domain.

“Faraday cage” refers to an electromagnetic shielding configuration that provides an electrical path to ground for unwanted electromagnetic radiation, thereby quieting an electromagnetic environment.

A “signal-analysis score” refers to a score based on analysis of a time-domain signals by one of the scoring algorithms discussed below.

An “optimized agent-specific time-domain signal” refers to a time-domain signal having a maximum or near-maximum signal-analysis score.

“In vitro system” refers to a biochemical system having of one or more biochemical components, such as nucleic acid or protein components, including receptors and structural proteins isolated or derived from a virus, bacteria, or multicellular plant or animal. An in vitro system typically is a solution or suspension of one or more isolated or partially isolated in vitro components in an aqueous medium, such as a physiological buffer. The term also refers to a cell culture system containing bacterial or eukaryotic cells in a culture medium.

“Mammalian system” refers to a mammal, include a laboratory animal such as mouse, rat, or primate that may serve as a model for a human disease, or a human patient.

“A chemical, biochemical, or biological system” refers to a system capable of evincing an agent-specific response to transduction by an agent-specific signal, or an agent-specific response in response to addition of a signal-exposed aqueous composition of the invention. A chemical or biochemical system may include, for example, one or more chemical or biochemical components in an aqueous solution or suspension, or a cell-free system of cellular components. A biological system may include an in vitro cell-culture system or in vivo animal system.

“Agent-specific effect” refers to an effect observed when a chemical, biochemical, or biological system is exposed to a chemical or biochemical agent (effector). Examples of agent-specific in vitro effects on a biological in vitro system include, for example, a change in the state of aggregation of components of the system, the binding the an agent to a target, such as a receptor, and the change in growth or division of cells in culture.

A “selected magnetic field strength within q range between 1 G and 10⁻⁸ G” refers to the magnetic field strength produced by one or more electromagnetic coils to which is applied a time-domain signal current calculated to produce a magnetic field strength that is either a selected constant field strength between 1 G and 10⁻⁸ G, or the magnetic field produced by a series of signal currents calculated to produce a plurality of incremental field strengths within a selected range, at least a portion of which is within the range 1 G and 10⁻⁸ G, e.g., 10⁻⁵ to 10⁻⁹ G.

“An aqueous medium” refers to a liquid medium having a water phase suitable to accept an agent-specific signal, and includes water, salt solutions, emulsions, foams, gels, suspensions, and pastes. The aqueous medium may contain up to 50 weight percent of other solvents, such as ethanol. Exemplary aqueous media include sterile, ultrapure water or physiological saline, e.g., a buffered isotonic solution suitable for parenteral injection in a patient, and may additionally contain ethanol at a volume concentration of between 0.1 and 50%, such that the aqueous medium composition, when formulated or diluted for intravenous administration, contains between 0.1 to 10, preferably 0.5 to 5 volume percent ethanol. The presence of ethanol may act to enhance the stability of the composition. Aqueous-medium suspensions may include aqueous suspensions of microparticles or nanoparticles, such as lipoosomes, as described below.

A “mechanically disrupted aqueous medium” refers to an aqueous medium that has been subjected to mechanical disruption forces, such as by vortexing, e.g., vigorous vortexing for 10-30 seconds, tapping, or sonication. The disruptive force may be applied in the absence of a gas, but is preferably carried out in the presence of a gas such as air.

An “interfacial aqueous medium” refers to an aqueous medium formulated or processed to contain gas microbubbles or other structures, such as suspended particles, capable of providing centers of gas/liquid or solid/liquid interfaces at which water structures can form, when an aqueous medium containing the interfaces is exposed to a love-frequency agent-specific signal, in accordance with the invention. A gas interfacial aqueous medium is produced, for example, by bubbling a gas, e.g., air, oxygen, nitrogen, or argon, into an aqueous medium, or by mechanical agitating an aqueous medium, e.g., by vortexing, sonication, or other mechanical agitation in the presence of the gas, or by the addition of gas nanoparticles or gas-producing compounds, such as bicarbonate salts. The amount and stability of gas bubbles in an aqueous medium may be enhanced by addition of additives, such as pharmaceutically acceptable surfactants. One interfacial aqueous medium is a foam formed by foaming an aqueous medium containing a foam-forming polymer, such as a cellulose, as described in U.S. Pat. Nos. 7,011,702 and 6,262,128. A number of suspendable nanoparticles, such sonicated lipid particles in an oil-in-water emulsion, latex particle, protein-shell gas- or liquid-filled nanoparticles, and liposomes or lipid vesicles, are well known. A suspension of liposomes, e.g., large unilamellar liposomes, can be prepared according to known methods, such as described in U.S. Pat. Nos. 5,030,453 and 5,059,421, and references cited therein. Liposome-encapsulated hydrogels can be formed as described in U.S. Pat. No. 7,619,565.

A “mechanically disrupted, interfacial aqueous medium” is both mechanically disrupted and contains interfacial gas bubbles, and may be formed, for example, by vigorous vortexing in the presence of air at normal atmospheric pressure.

“Paclitaxel or analog thereof” refers a class of diterpine compounds produced by the plants of the genus Taxus, and chemical analogs thereof, including but not limited to paclitaxel, docetaxel, larotaxel, ortataxel and tesetaxel.

A “taxane-like compound” or “paclitaxel-like compound” refers to a compound that operate through a mechanism of action involving enhancing tubulin polymer formation and/or stabilizing formed tubulin polymer. Included in this definition are taxane compounds and epithilones, such as epothilones A to F, and analogs thereof, such as ixabepilone (epithilone B). These compounds are known to bind to the αβ-tubulin heterodimer subunit, like taxanes, and once bound, decrease the rate of dissociation of the heterodimers, Epothilone B has also been shown to induce tubulin polymerization into microtubules without the presence of GTP. This is caused by formation of microtubule bundles throughout the cytoplasm. Finally, epothilone B also causes cell cycle arrest at the G2-M transition phase, thus leading to cytotoxicity and eventually cell apoptosis. (Balog, D. M.; Meng, D.; Kamanecka, T.; Bertinato, P. Su, D.-S.; Sorensen, E. J.; Danishefsky, S. J. Angew, Chem, 1996, 108, 2976. Some endotoxin-like properties known from paclitaxel, however, like activation of macrophages synthesizing inflammatory cytokines and nitric oxide, are not observed for epothilone B.

A “therapeutic oligonucleotide” refers to a single-stranded (ss) or double-stranded (ds) RNA, DNA, or an oligonucleotide analog having a modified backbone or bases, that can function in a therapeutic role when present in a cellular environment, typically by inhibiting or activating the expression of one or more selected cellular proteins. A therapeutic oligonucleotide is typically 10-50 nucleotide bases in length, preferably 15-30 bases, and may function, for example, as (i) a single-stranded antisense compound capable of binding to a complementary sequence DNA or RNA to inhibit transcription of RNA from DNA or translation of RNA into proteins, or to induce transcript processing errors, such as exon skipping, (ii) a double-stranded DNA that functions as a small interfering RNA (siRNA) to interfere with expression of a specific gene, (iii) small double-stranded RNA that functions to activate gene expression, and (iv) single-stranded micro RNAs that function as gene silencers in selected target mRNAs. Exemplary therapeutic oligonucleotides include: GAPDH antisense RNA and PCSK9 antisense RNA, both described below.

“Taxane signal” or “paclitaxel signal” refers to a low-frequency time-domain signal recorded for a taxane compound, e.g., paclitaxel, and which is capable of inducing taxane-like specific effects under conditions of exposure to the signal, as detailed herein.

A “therapeutic oligonucleotide signal” refers to a low-frequency time-domain signal recorded for a therapeutic oligonucleotide compound, e.g., GAPDH antisense RNA or PCSK9 antisense RNA.

“Water signal” refers to low-frequency time-domain signal recorded for a sample of pure water, under conditions identical to those used for recording an agent signal, such as a taxane signal.

“Water exposed to a taxane signal” refers to an aqueous medium that has been exposed to a taxane signal under conditions detailed herein.

“Water exposed to a therapeutic oligonucleotide signal” refers to an aqueous medium that has been exposed to a therapeutic oligonucleotide signal under conditions detailed herein.

“Water exposed to a water signal” or “water exposed to white noise” refers to a sample of water, e.g., ultrapure water, that has been exposed to a water or white noise signal, respectively, under conditions detailed herein.

“Transducing” a chemical, biochemical, or biological system refers to exposing the system to an agent-specific signal, and achieving thereby, an agent-specific effect in the system. One model transduction system described below is a cell-culture system whose cells can respond to the agent-specific signal, e.g., by reduced growth rate, or stimulation or inhibition of expression of a selected cellular component.

“Exposing” an aqueous medium to an agent-specific signal means placing the medium in an electromagnetic field generated by a low-frequency signal recorded from the agent, in accordance with the invention.

An aqueous composition is said to “mimic” the action of a chemical or biochemical agent capable producing an agent-specific effect in a chemical, biochemical, or biological system, if the composition is effective to produce at least one agent-specific effect on the system.

The “activity of a composition, expressed in terms of the concentration of a given chemical or biological agent,” means that the composition has the same activity, with respect to at least one effect of the chemical or biological agent, as a solution or suspension of the agent at the given concentration of the agent. Thus, for example, a composition having a paclitaxel activity, expressed in terms of paclitaxel concentration, of between 0.01 and 10 μM, means that the composition has the same activity, in terms of its ability to inhibit as a suspension of paclitaxol-responsive cancer cells, or in its ability to inhibit the growth of a taxol-responsive tumor in an animal, as a solution of paclitaxel at a concentration between 0.01 and 10 μM.

### II. Apparatus for Generating Agent-Specific Signals

A recording apparatus for producing time-domain signals from samples of a selected agent is detailed in co-owned PCT application WO2008/063654, which is incorporated herein Certain preferred embodiments of the apparatus and scoring algorithms are described below.

The apparatus is used by placing a sample within the magnetically shielded faraday cage in close proximity to the coil that generates the stimulus signal and the gradiometer that measures the response. A stimulus signal is injected through the stimulus coil, and this signal may be modulated until a desired optimized signal is produced. The molecular electromagnetic response signal, shielded from external interference by the faraday cage and the field generated by the stimulus coil, is then detected and measured by the gradiometer and SQUID. The signal is then amplified and transmitted to any appropriate recording or measuring equipment.

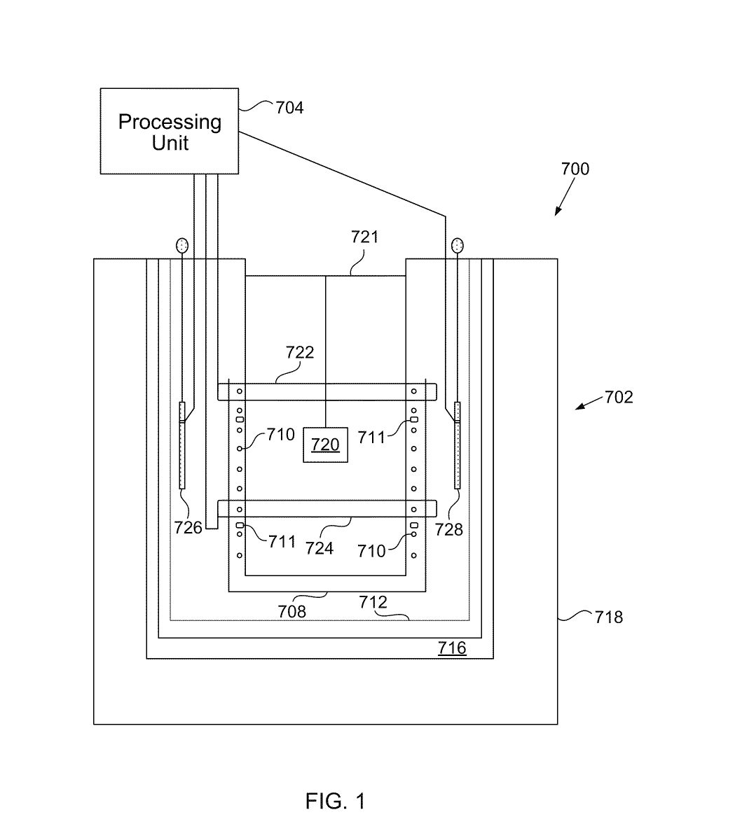

FIG. 1 shows one embodiment of an apparatus for electromagnetic emission detection and a processing system, Apparatus 700 includes a detection unit 702 coupled to a processing unit 704. Although the processing unit 704 is shown external to the detection unit 702, at least a part of the processing unit can be located within the detection unit.

The detection unit 702, which is shown in a cross-sectional view in FIG. 1, includes multiple components nested or concentric with each other. A sample chamber or faraday cage 706 is nested within a metal cage 708. Each of the sample chamber 706 and the metal cage 708 can be comprised of aluminum material. The sample chamber 706 can be maintained in a vacuum and may be temperature controlled to a preset temperature. The metal cage 708 is configured to function as a low pass filter.

Between the sample chamber 706 and the metal cage 708 and encircling the sample chamber 706 are a set of parallel heating coils or elements 710. One or more temperature sensor 711 is also located proximate to the heating elements 710 and the sample chamber 706. For example, four temperature sensors may be positioned at different locations around the exterior of the sample chamber 706. The heating elements 710 and the temperature sensor(s) 711 may be configured to maintain a certain temperature inside the sample chamber 706.

A shield 712 encircles the metal cage 708. The shield 712 is configured to provide additional magnetic field shielding or isolation for the sample chamber 706. The shield 712 can be comprised of lead or other magnetic shielding materials. The shield 712 is optional when sufficient shielding is provided by the sample chamber 706 and/or the metal cage 708.

Surrounding the shield 712 is a cryogen layer 716 with G10 insulation. The cryogen may be liquid helium. The cryogen layer 716 (also referred to as a cryogenic Dewar) is at an operating temperature of 4 degrees Kelvin, Surrounding the cryogen layer 716 is an outer shield 716. The outer shield 718 is comprised of nickel alloy and is configured to be a magnetic shield. The total amount of magnetic shielding provided by the detection unit 702 is approximately −100 dB, −100 dB, and −120 dB along the three orthogonal planes of a Cartesian coordinate system.

The various elements described above are electrically isolated from each other by air gaps or dielectric barriers (not shown). It should also be understood that the elements are not shown to scale relative to each other for ease of description.

A sample holder 720 can be manually or mechanically positioned within the sample chamber 706. The sample holder 720 may be lowered, raised, or removed from the top of the sample chamber 706. The sample holder 720 is comprised of a material that will not introduce Eddy currents and exhibits little or no inherent molecular rotation. As an example, the sample holder 720 can be comprised of high quality glass or Pyrex.

The detection unit 702 is configured to handle solid, liquid, or gas samples. Various sample holders may be utilized in the detection unit 702. For example, depending on the size of the sample, a larger sample holder may be utilized. As another example, when the sample is reactive to air, the sample holder can be configured to encapsulate or form an airtight seal around the sample. In still another example, when the sample is in a gaseous state, the sample can be introduced inside the sample chamber 706 without the sample holder 720. For such samples, the sample chamber 706 is held at a vacuum. A vacuum seal 721 at the top of the sample chamber 706 aids in maintaining a vacuum and/or accommodating the sample holder 720.

A sense coil 722 and a sense coil 724, also referred to as detection cons, are provided above and below the sample holder 720, respectively. The coil windings of the sense coils 722, 724 are configured to operate in the direct current (DC) to approximately 50 kilohertz (kHz) range, with a center frequency of 25 kHz and a self-resonant frequency of 8.8 MHz. The sense coils 722, 724 are in the second derivative form and are configured to achieve approximately 100% coupling. In one embodiment, the coils 722, 724 are generally rectangular in shape and are held in place by G10 fasteners. The coils 722, 724 function as a second derivative gradiometer.

Helmholtz coils 726 and 728 may be vertically positioned between the shield 712 and the metal cage 708, as explained herein. Each of the coils 726 and 728 may be raised or lowered independently of each other. The coils 726 and 728, also referred to as magnetic-field stimulus generation coils, are at room or ambient temperature. The noise generated by the coils 726, 728 is approximately 0.10 Gauss.

The degree of coupling between the emissions from the sample and the coils 722, 724 may be changed by repositioning the sample holder 720 relative to the coils 722, 724, or by repositioning one or both of the coils 726, 728 relative to the sample holder 720.

The processing unit 704 is electrically coupled to the coils 722, 724, 726, and 728. The processing unit 704 specifies the magnetic-field stimulus, e.g., Gaussian white noise stimulus to be injected by the coils 726, 728 to the sample. The processing unit 104 also receives the induced voltage at the coils 722, 724 from the sample's electromagnetic emissions mixed with the injected magnetic-field stimulus.

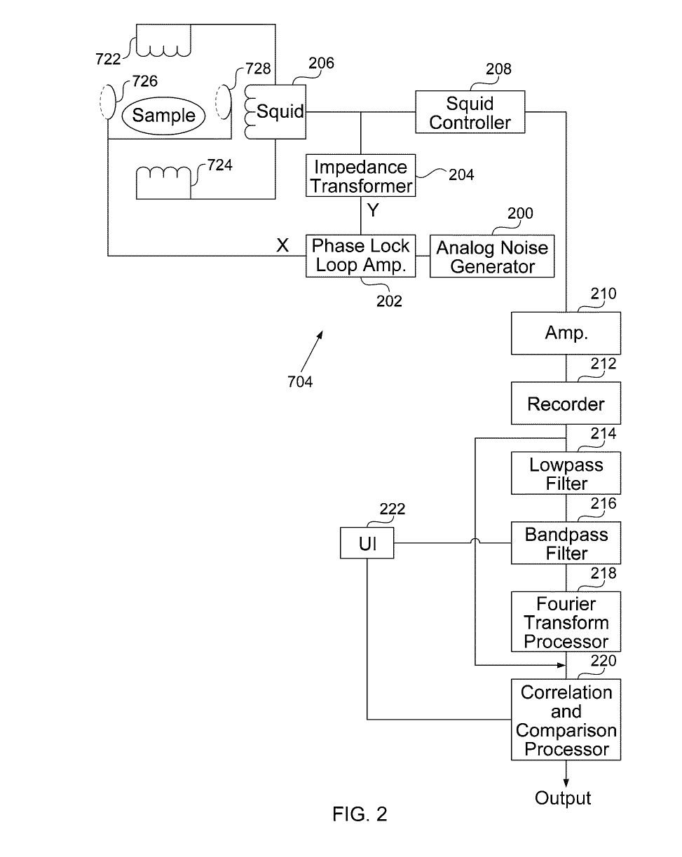

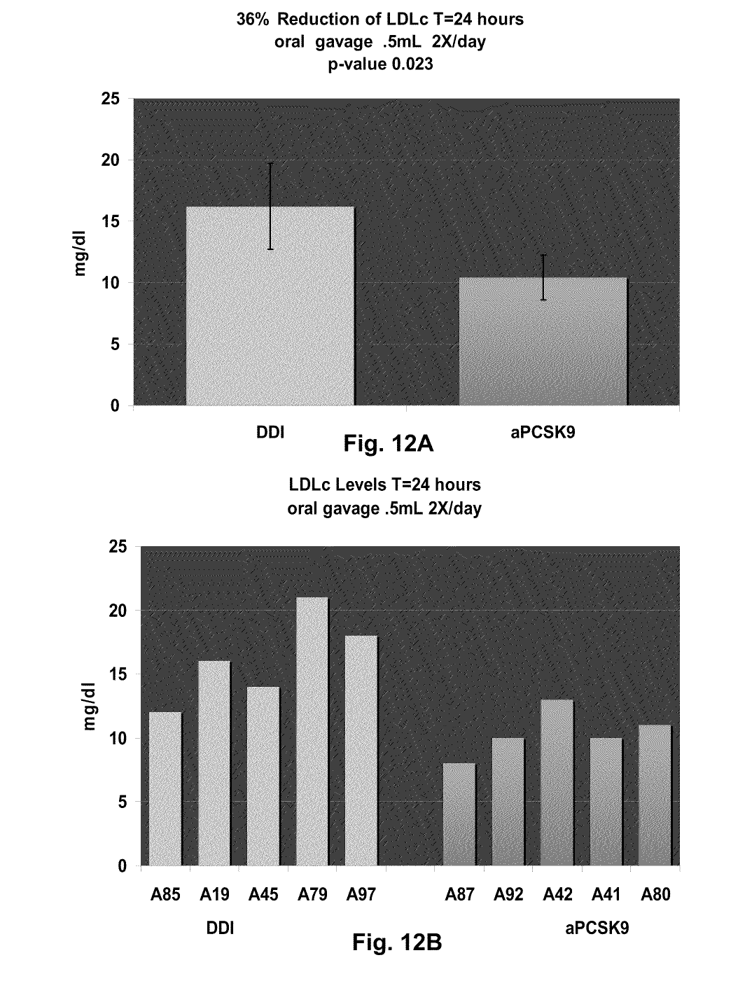

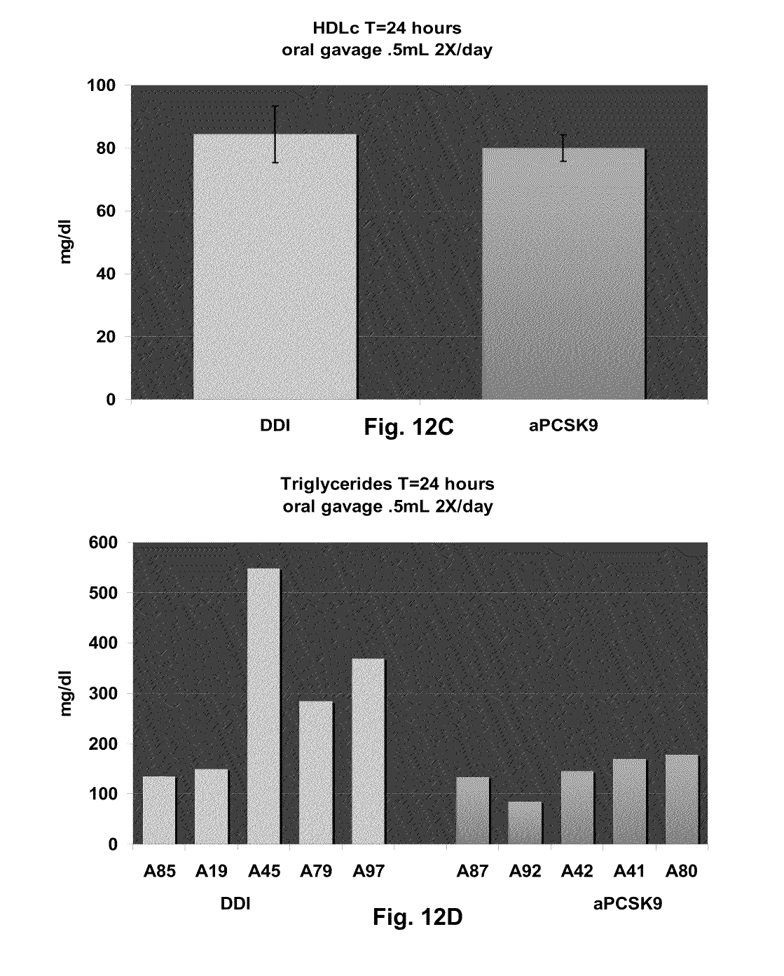

FIG. 2 is a block diagram of the processing unit shown at 704 in FIG. 12. A dual phase lock-in amplifier 202 is configured to provide a first magnetic-field signal (e.g., “x” or noise stimulus signal) to the coils 726, 728 and a second magnetic-field signal (e.g., “y” or noise cancellation signal) to a noise cancellation coil of a superconducting quantum interference device (SQUID) 206. The amplifier 202 is configured to lock without an external reference and may be a Perkins Elmer model 7265 DSP lock-in amplifier. This amplifier works in a “virtual mode,” where it locks to an initial reference frequency, and then removes the reference frequency to allow it to run freely and lock to “noise.”

A magnetic-field stimulus generator, such as an analog Gaussian white noise stimulus generator 200 is electrically coupled to the amplifier 202. The generator 200 is configured to generate a selected magnetic-field stimulus, e.g., analog Gaussian white noise stimulus at the coils 726, 728 via the amplifier 202. As an example, the generator 200 may be a model 1380 manufactured by General Radio.

An impedance transformer 204 is electrically coupled between the SQUID 206 and the amplifier 202. The impedance transformer 204 is configured to provide impedance matching between the SQUID 206 and amplifier 202.

The SQUID 206 is a low temperature direct element SQUID. As an example, the SQUID 206 may be a model LSQ/20 LTS dC SQUID available form Tristan Technologies, Inc (San Diego, Calif.,) Alternatively, a high temperature or alternating current SQUID can be used. The coils 722, 724 (e.g., gradiometer) and the SQUID 206 (collectively referred to as the SQUID/gradiometer detector assembly) combined has a magnetic field measuring sensitivity of approximately 5 microTesla/√Hz. The induced voltage in the coils 722, 724 is detected and amplified by the SQUID 206. The output of the SQUID 206 is a voltage approximately in the range of 0.2-0.8 microVolts.

The output of the SQUID 206 is the input to a SQUID controller 208. The SQUID controller 208 is configured to control the operational state of the SQUID 206 and further condition the detected signal. As an example, the SQUID controller 208 may be an iMC-303 iMAG multi-channel SQUID controller manufactured by Tristan Technologies, Inc.

The output of the SQUID controller 208 is inputted to an amplifier 210. The amplifier 210 is configured to provide a gain in the range of 0-100 dB. A gain of approximately 20 dB is provided when noise cancellation node is turned on at the SQUID 206. A gain of approximately 50 dB is provided when the SQUID 206 is providing no noise cancellation.

The amplified signal is inputted to a recorder or storage device 212. The recorder 212 is configured to convert the analog amplified signal to a digital signal and store the digital signal. In one embodiment, the recorder 212 stores 8600 data points per Hz and can handle 2.46 Mbits/sec. As an example, the recorder 212 may be a Sony digital audiotape (DAT) recorder. Using a DAT recorder, the raw signals or data sets can be sent to a third party for display or specific processing as desired.

A lowpass filter 214 filters the digitized data set from the recorder 212. The lowpass filter 214 is an analog filter and may be a Butterworth filter. The cutoff frequency is at approximately 50 kHz.

A bandpass filter 216 next filters the filtered data sets. The bandpass filter 216 is configured to be a digital filter with a bandwidth between DC to 50 kHz. The bandpass filter 216 can be adjusted for different bandwidths.

The output of the bandpass filter 216 is the input to a Fourier transformer processor 218. The Fourier transform processor 218 is configured to convert the data set, which is in the time domain, to a data set in the frequency domain. The Fourier transform processor 218 performs a Fast Fourier Transform (FFT) type of transform.

The Fourier transformed data sets are the input to a correlation and comparison processor 220. The output of the recorder 212 is also an input to the processor 220. The processor 220 is configured to correlate the data set with previously recorded data sets, determine thresholds, and perform noise cancellation (when no noise cancellation is provided by the SQUID 206). The output of the processor 220 is a final data set representative of the spectrum of the sample's molecular low frequency electromagnetic emissions.

A user interface (UI) 222, such as a graphical user interface (GUI), may also be connected to at least the filter 216 and the processor 220 to specify signal processing parameters. The filter 216, processor 218, and the processor 220 can be implemented as hardware, software, or firmware. For example, the filter 216 and the processor 218 may be implemented in one or more semiconductor chips. The processor 220 may be software implemented in a computing device.

This amplifier works in a “virtual mode,” where it locks to an initial reference frequency, and then removes the reference frequency to allow it to run freely and lock to “noise.” The analog noise generator (which is produced by General Radio, a truly analog noise generator) requires 20 dB and 45-dB attenuation for the Helmholtz and noise cancellation coil, respectively.

The Helmholtz coil may have a sweet spot of about one cubic inch with a balance of 1/100 of a percent. In an alternative embodiments, the Helmholtz coil may move both vertically, rotationally (about the vertical axis), and from parallel to spread apart in a pie shape. In one embodiment, the SQUID, gradiometer, and driving transformer (controller) have values of 1.8, 1.5 and 0.3 micro-Henrys, respectively. The Helmholtz coil may have a sensitivity of 0.5 Gauss per amp at the sweet spot.

Approximately 10 to 15 microvolts may be needed for a stochastic response. By injecting Gaussian white noise stimulus, the system has raised the sensitivity of the SQUID device. The SQUID device had a sensitivity of about 5 femtotesla without the noise. This system has been able to improve the sensitivity by 25 to 35 dB by injecting noise and using this stochastic resonance response, which amounts to nearly a 1,500% increase.

After receiving and recording signals from the system, a computer, such as a mainframe computer, supercomputer or high-performance computer does both pre and post processing, such by employing the Autosignal software product by Systat Software of Richmond Calif., for the pre-processing, while Flexpro software product does the post-processing, Flexpro is a data (statistical) analysis software supplied by Dewetron, Inc. The following equations or options may be used in the Autosignal and Flexpro products.

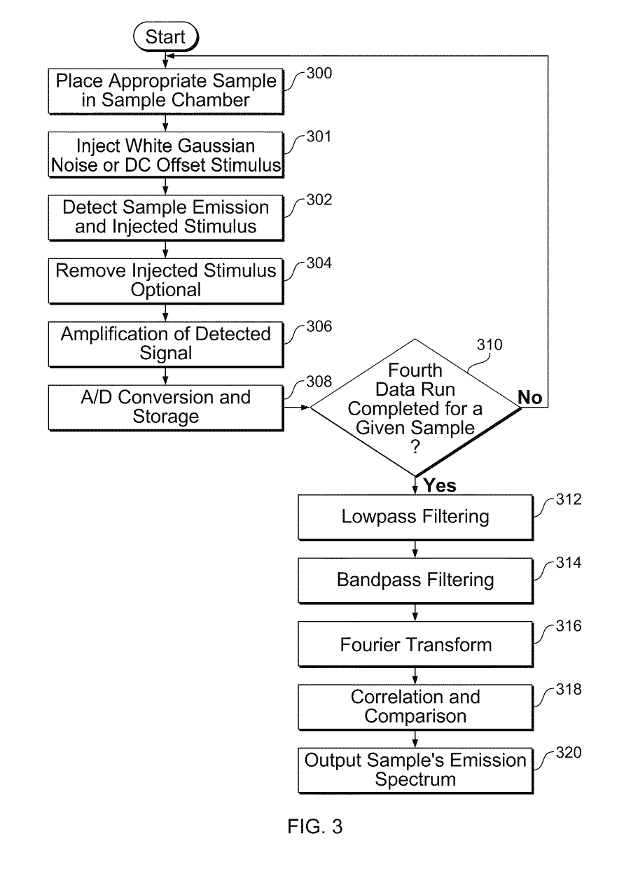

A flow diagram of the signal detection and processing performed by the apparatus is shown in FIG. 3. When a sample is of interest, typically at least four signal detections or data runs are performed: a first data run at a time t₁ without the sample, a second data run at a time t₂ with the sample, a third data run at a time t₃ with the sample, and a fourth data run at a time t₄ without the sample. Performing and collecting data sets from more than one data run increases accuracy of the final (e.g., correlated) data set. In the four data runs, the parameters and conditions of the system are held constant (e.g., temperature, amount of amplification, position of the coils, the Gaussian white noise and/or DC offset signal, etc.).

At block 300, the appropriate sample (or if its a first or fourth data run, no sample), is placed in the apparatus, e.g., apparatus 700. A given sample, without injected Gaussian white noise or DC-offset stimulus, emits electromagnetic emissions in the DC-50 kHz range at an amplitude equal to or less than approximately 0.001 microTesla. To capture such low emissions, Gaussian white noise stimulus and/or DC offset is injected at block 301.

At block 302, the coils 722, 724 detect the induced voltage representative of the sample's emission and the injected magnetic stimulus. The induced voltage comprises a continuous stream of voltage values (amplitude and phase) as a function of time for the duration of a data run. A data run can be 2-20 minutes in length and hence, the data set corresponding to the data run comprises 2-20 minutes of voltage values as a function of time.

At block 304, the injected magnetic stimulus is cancelled as the induced voltage is being detected. This block is omitted when the noise cancellation feature of the SQUID 206 is turned off.

At block 306, the voltage values of the data set are amplified by 20-50 dB, depending on whether noise cancellation occurred at the block 304. And at-block 308, the amplified data set undergoes analog to digital (A/D) conversion and is stored in the recorder 212. A digitized data set can comprise millions of rows of data.

After the acquired data set is stored, at a block 310 a check is performed to see whether at least four data runs for the sample have occurred (e.g., have acquired at least four data sets). if four data sets for a given sample have been obtained, then lowpass filtering occurs at block 312. Otherwise, the next data run is initiated (return to the block 300).

After lowpass filtering (block 312) and bandpass filtering (at a block 314) the digitized data sets, the data sets are converted to the frequency domain at a Fourier transform block 316.

Next, at block 318, like data sets are correlated with each other at each data point. For example, the first data set corresponding to the first data run (e.g., a baseline or ambient noise data run) and the fourth data set corresponding to the fourth data run (e.g., another noise data run) are correlated to each other. If the amplitude value of the first data set at a given frequency is the same as the amplitude value of the fourth data set at that given frequency, then the correlation value or number for that given frequency would be 1.0. Alternatively, the range of correlation values may be set at between 0-100. Such correlation or comparison also occurs for the second and third data runs (e.g., the sample data runs). Because the acquired data sets are stored, they can be accessed at a later time as the remaining data runs are completed.

Predetermined threshold levels are applied to each correlated data set to eliminate statistically irrelevant correlation values. A variety of threshold values may be used, depending on the length of the data runs (the longer the data runs, greater the accuracy of the acquired data) and the likely similarity of the sample's actual emission spectrum to other types of samples. In addition to the threshold levels, the correlations are averaged. Use of thresholds and averaging correlation results in the injected Gaussian white noise stimulus component becoming very small in the resulting correlated data set.

Once the two sample data sets have been refined to a correlated sample data set and the two noise data sets have been refined to a correlated noise data set, the correlated noise data set is subtracted from the correlated sample data set. The resulting data set is the final data set (e.g., a data set representative of the emission spectrum of the sample) (block 320).

Since there can be 8600 data points per Hz and the final data set can have data points for a frequency range of DC-50 kHz, the final data set can comprise several hundred million rows of data. Each row of data can include the frequency, amplitude, phase, and a correlation value,

### III. Method of Identifying Optimal Time-Domain Signals for Transduction

The agent-specific signals produced in accordance with the apparatus and methods described above may be further selected for optimal effector activity, when used to transduce, for example, an in vitro or mammalian system. As detailed in co-owned PCT application WO2008/063654 A3, agent-dependent signal features in a time-domain signal obtained for a given agent can be optimized by recording time-domain signals for the sample over a range of magnetic-field stimulus conditions, e.g., different voltage levels for Gaussian white noise stimulus amplitudes and/or DC offsets. The recorded signals are then processed to reveal signal features, and one or more time domain signals having an optimal signal-analysis score, as detailed below, are selected. The selection of optimized or near-optimized time-domain signals is useful because it has been found that transducing a system, such as an in vitro biological system, or exposing an aqueous medium to an optimized time-domain signal gives a stronger and more predictable response than with a non-optimized time-domain signal. That is, selecting an optimized (or near-optimized) time-domain signal is useful in achieving reliable, detectable sample effects when a target system is transduced by the sample signal, or when an aqueous medium is exposed to the signal.

Agent-specific signals are typically recorded by first dissolving or suspending the selected agent, e.g., biological or biochemical agent, in a suitable aqueous medium, e.g., purified water, as illustrated below for oligonucleotide agents. For agents that have poor solubility, e.g., taxanes, the agent may be suspended in a suitable vehicle, such as Cremophor EL™ or other vehicle containing suitable solubilizing or suspending agents, as illustrated below for both paclitaxel.

The concentration of the agent is typically adjusted to between 10⁻³ to 10⁻²⁴ M, with a preferred range between about 10⁻¹⁰ to 10⁻¹⁶ μM. The sample may be treated, prior to recording, to form one of: (i) a mechanically disrupted sample medium, (ii) an interfacial sample medium containing gas bubbles, and (iii) a mechanically disrupted interfacial sample medium containing gas bubbles. Treatment for mechanical disruption may be, for example, by vigorous vortexing for 5-30 seconds, which if carried out in the presence of air, also results in a interfacial medium having suspended gas bubbles. The sample is typically recorded at between 4-37° C., preferably room temperature, i.e., about 24° C.

In general, the range of injected white noise and DC offset voltages applied to the sample are such as to produce a calculated magnetic field at the sample container of between 0 to 1 G (Gauss), or alternatively, the injected noise stimulus is preferably between about 30 to 35 decibels above the molecular electromagnetic emissions sought to be detected, e.g., in the range 70-80-dbm. The number of samples that are recorded, that is, the number of noise-level intervals over which time-domain signals are recorded may vary from 10-100 or more, typically, and in any case, at sufficiently small intervals so that a good optimum signal can be identified. For example, the power gain of the noise generator level can be varied over 50 20 mV intervals.

Alternatively, stimulus signals other than Gaussian white noise and/or DC offset can be used for optimization of the recorded time-domain signal. Examples of such signals include scanning a range of sine wave frequencies, a square wave, time-series data containing defined non-linear structure, or the SQUID output itself. These signals may themselves be pulsed between off and on states to further modify the stimulus signal. The white noise naturally generated by the magnetic shields may also be used as the source of the stimulus signal.

Above-cited PCT application WO 2008/063654 describes five methods for scoring the time-domain signals produced as above: (A) a histogram bin method, (B) generating an FFT of autocorrelated signals, (C) averaging of FFTs, (D) use of a cross-correlation threshold, and (E) phase-space comparison. Of these, the most successful predictors of effective transduction signals have been the histogram bin method (A), and enhanced autocorrelation (EAC) method (B). The two preferred methods are discussed below.

### A. Histogram Method of Generating Spectral Information

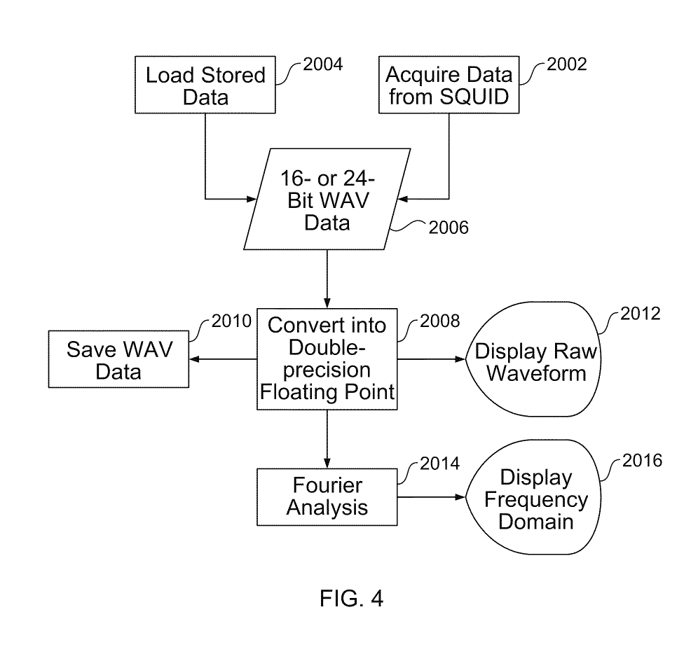

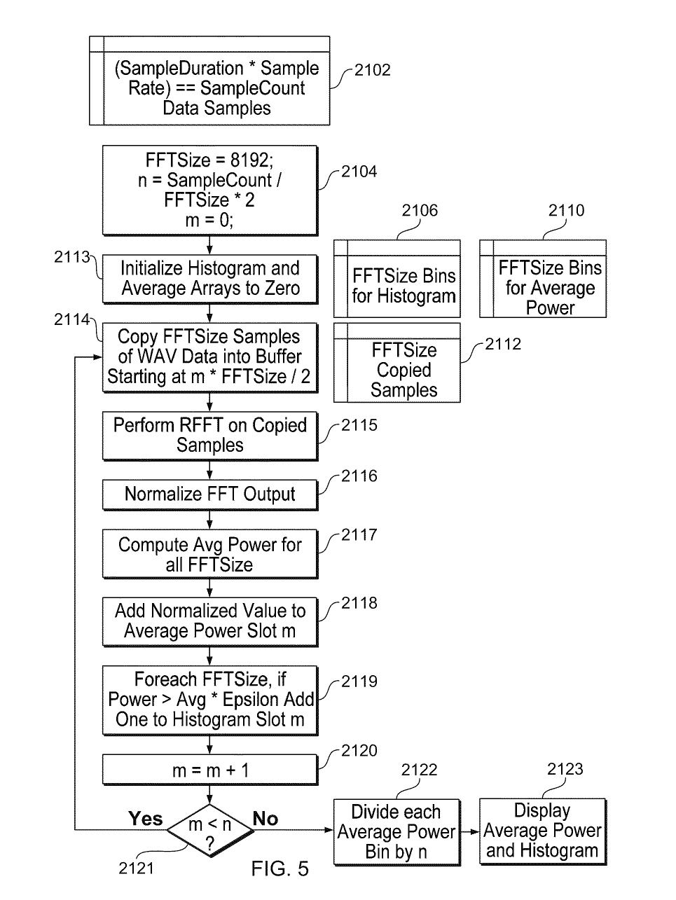

FIG. 4 is a high level data flow diagram in the histogram method for generating spectral information. Data acquired from the SQUID (box 2002) or stored data (box 2004) is saved as 16 or 24 bit WAV data (box 2006), and converted into double-precision floating point data (box 2008). The converted data may be saved (box 2010) or displayed as a raw waveform (box 2012). The converted data is then passed to the algorithm described below with respect to FIG. 5, and indicated by the box 2014 labeled Fourier Analysis. The histogram can be displayed at 2016.

FIG. 5 shows the general flow of the histogram scoring algorithm. The time-domain signals are acquired from an ADC (analog/digital converter) and stored in the buffer indicated at 2102. This sample is SampleDuration seconds long, and is sampled at SampleRate samples per second, thus providing SampleCount (SampieDuration*SampleRate) samples. The FrequencyRange that can be recovered from the signal is defined as half the SampleRate, as defined by Nyquist. Thus, if a time-series signal is sampled at 10,000 samples per second, the FrequencyRange will be 0 Hz to 5 kHz. One Fourier algorithm that may be used is a Radix 2 Real Fast Fourier Transform (RFFT), which has a selectable frequency domain resolution (FFTSize) of powers of two up to 2¹⁶. An FFTSize of 8192 is selected, to provide provides enough resolution to have at least one spectrum bin per Hertz as long as the FrequencyRange stays at or below 8 kHz. The SampleDuration should be long enough such that SampleCount>(2*)FFTSize*10 to ensure reliable results.

Since this FFT can only act on FFTSize samples at a time, the program must perform the FFT on the samples sequentially and average the results together to get the final spectrum. If one chooses to skip FFTSize samples for each FFT, a statistical error of 1/FFTSizê0.5 is introduced. If, however, one chooses to overlap the FFT input by half the FFTSize, this error is reduced to 1/(0.81*2*FFTSize)̂0.5. This reduces the error from 0.0110485435 to 0.0086805556. Additional information about errors and correlation analyses in general, consult Bendat & Piersol, “Engineering Applications of Correlation and Spectral Analysis”, 1993.

Prior to performing the FFT on a given window, a data tapering filter may be applied to avoid spectral leakage due to sampling aliasing. This filter can be chosen from among Rectangular (no filter), Hamming, Hanning, Bartlett, Blackman and Blackman/Harris, as examples.

In an exemplary method, and as shown in box 2104, we have chosen 8192 for the variable FFTSize, which will be the number of time-domain samples we operate on at a time, as well as the number of discrete frequencies output by the FFT. Note that FFTSize=8192 is the resolution, or number of bins in the range which is dictated by the sampling rate. The variable n, which dictates how many discrete RFFT's (Real FFT's) performed, is set by dividing the SampleCount by FFTSize*2, the number of FFT bins. In order for the algorithm to generate sensible results, this number n should be at least 10 to 20 (although other valves are possible), where more may be preferred to pick up weaker signals. This implies that for a given SampleRate and FFTSize, the SampleDuration must be long enough. A counter m, which counts from 0 to n, is initialized to zero, also as shown in box 2104.

The program first establishes three buffers: buffer 2108 for FFTSize histogram bins, that will accumulate counts at each bin frequency; buffer 2110 for average power at each bin frequency, and a buffer 2112 containing the FFTSize copied samples for each m.

The program initializes the histograms and arrays (box 2113) and copies FFTSize samples of the wave data into buffer 2112, at 2114, and performs an RFFT on the wave data (box 2115). The FFT is normalized so that the highest amplitude is 1 (box 2116) and the average power for all FFTSize bins is determined from the normalized signal (box 2117). For each bin frequency, the normalized value from the FFT at that frequency is added to each bin in buffer 2108 (box 2118).

In box 2119 the program then looks at the power at each bin frequency, relative to the average power calculated from above. If the power is within a certain factor epsilon (between 0 and 1) of the average power, then it is counted and the corresponding bin is incremented in the histogram buffer at 16. Otherwise it is discarded.

Note that the average power it is comparing to is for this FFT instance only. An enhanced, albeit slower algorithm might take two passes through the data and compute the average over all time before setting histogram levels. The comparison to epsilon helps to represent a power value that is significant enough for a frequency bin. Or in broader terms, the equation employing epsilon helps answer the question, “is there a signal at this frequency at this time?” If the answer is yes, it could due be one of two things: (1) stationary noise which is landing in this bin just this one time, or (2) a real low level periodic signal which will occur nearly every time. Thus, the histogram counts will weed out the noise hits, and enhance the low level signal hits. So, the averaging and epsilon factor allow one to select the smallest power level considered significant.

Counter m is incremented at box 2120, and the above process is repeated for each n set of WAV data until m is equal to n (box 2121). At each cycle, the average power for each bin is added to the associated bin at 2118, and each histogram bin is incremented by one when the power amplitude condition at 2114 is met.

When all n cycles of data have been considered, the average power in each bin is determined by dividing the total accumulated average power in each bin by n, the total number of cycles (box 2122) and the results displayed (box 2123). Except where structured noise exists, e.g., DC=0 or at multiples of 60 Hz, the average power in each bin will be some relatively low number.

The relevant settings in this method are noise stimulus gain and the value of epsilon. This value determines a power value that will be used to distinguish an event over average value. At a value of 1, no events will be detected, since power will never be greater than average power. As epsilon approaches zero, virtually every value will be placed in a bin. Between 0 and 1, and typically at a value that gives a number of bin counts between about 20-50% of total bin counts for structured noise, epsilon will have a maximum “spectral character,” meaning the stochastic resonance events will be most highly favored over pure noise.

Therefore, one can systematically increase the power gain on the magnetic-field stimulus input, e.g., in 50 mV increments between 0 and 1 V, and at each power setting, adjust epsilon until a histogram having well defined peaks is observed. Where, for example, the sample being processed represents a 20 second time interval, total processing time for each different power and epsilon will be about 25 seconds. When a well-defined signal is observed, either the power setting or epsilon or both can be refined until an optimal histogram, meaning one with the largest number of identifiable peaks, is produced.

Under this algorithm, numerous bins may be filled and associated histogram rendered for low frequencies due to the general occurrence of noise (such as environmental noise) at the low frequencies. Thus, the system may simply ignore bins below a given frequency (e.g., below 1 kHz), but still render sufficient bin values at higher frequencies to determine unique signal signatures between samples.

Alternatively, since a purpose of the epsilon variable is to accommodate different average power levels determined in each cycle, the program could itself automatically adjust epsilon using a predefined function relating average power level to an optimal value of epsilon.

Similarly, the program could compare peak heights at each power setting, and automatically adjust the noise stimulus power setting until optimal peak heights or character is observed in the histograms.

Although the value of epsilon may be a fixed value for all frequencies, it is also contemplated to employ a frequency-dependent value for epsilon, to adjust for the higher value average energies that may be observed at low frequencies, e.g., DC to 1,000. A frequency-dependent epsilon factor could be determined, for example, by averaging a large number of low-frequency FFT regions, and determining a value of epsilon that “adjusts” average values to values comparable to those observed at higher frequencies.

### B. Enhanced Autocorrelation (EAC)

In a second preferred method for determining signal-analysis scores, time-domain signals recorded at a selected noise stimulus are autocorrelated, and a fast Fourier transform (FFT) of the autocorrelated signal is used to generate a signal-analysis plot, that is, a plot of the signal in the frequency domain. The FFTs are then used to score the number of spectral signals above an average noise level over a selected frequency range, e.g., DC to 1 kHz or DC to 8 kHz.

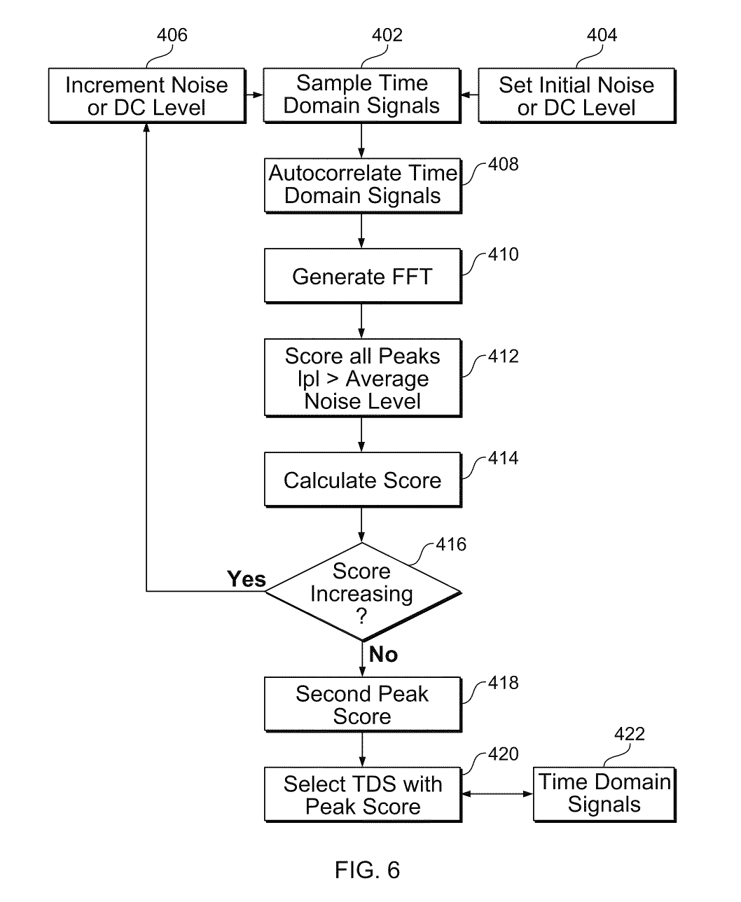

FIG. 6 is a flow diagram of steps carried out in scoring recorded time-domain signals according to this second embodiment. Time-domain signals are sampled, digitized, and filtered as above (box 402), with the gain on the magnetic-field stimulus level set to an initial level, as at 404. A typical time domain signal for a sample compound 402 is autocorrelated, at 408, using a standard autocorrelation algorithm, and the FFT of the autocorrelated function is generated, at 410, using a standard FFT algorithm.

An FFT plot is scored, at 412, by counting the number of spectral peaks that are statistically greater than the average noise observed in the autocorrelated FFT and the score is calculated at 414. This process is repeated, through steps 416 and 406, until a peak score is recorded, that is, until the score for a given signal begins to decline with increasing magnetic stimulus gain. The peak score is recorded, at 418, and the program or user selects, from the file of time-domain signals at 422, the signal corresponding to the peak score (box 420).

As above, this embodiment may be carried out in a manual mode, where the user manually adjusts the magnetic stimulus setting in increments, analyzes (counts peaks) from the FFT spectral plots by hand, and uses the peak score to identify one or more optimal time-domain signals. Alternatively, one or more aspects of the steps can be automated

In a related method, time-domain signals are converted by an FFT to the frequency domain, and pairs of frequency-domain signals, e.g., from the same sample, are cross-correlated. The cross-correlated signal may be further enhanced by cross-correlating with a second frequency-domain signal produced by cross-correlating a second pair of frequency-domain signals, e.g., from the same sample as above. Thus, four time-domain signals from the same sample are each converted to the frequency domain, and divided into two pairs, each of which are cross-correlated, then cross-correlated again to produce a final frequency-domain spectrum for that sample. The signal can then be scored by the number of peaks above a given noise threshold, and any of the four time-domain signals used in producing a top-scoring twice-cross-correlated signal may be employed in the transduction or exposing methods described below.

In one exemplary method, paclitaxel time-domain signals were obtained by recording low-frequency signals from a sample of paclitaxel suspended in CremophorEL™ 529 ml and anhydrous ethanol 69.74 ml to a final concentration of 6 mg/ml. The signals were recorded with injected DC offset, at noise level settings between 10 and 241 mV and in increments of 1 mV. A total of 241 time-domain signals over this injected-noise level range were obtained, and these were analyzed by an enhanced autocorrelation algorithm detailed above, yielding 8 time-domain paclitaxel-derived signals for further in vitro testing. One of these, designated signal M2(3), was selected as an exemplary paclitaxel signal effective in producing taxol-specific effects in biological response systems (described below), and when used for producing paclitaxel-specific aqueous compositions in accordance with the invention, also as described below.

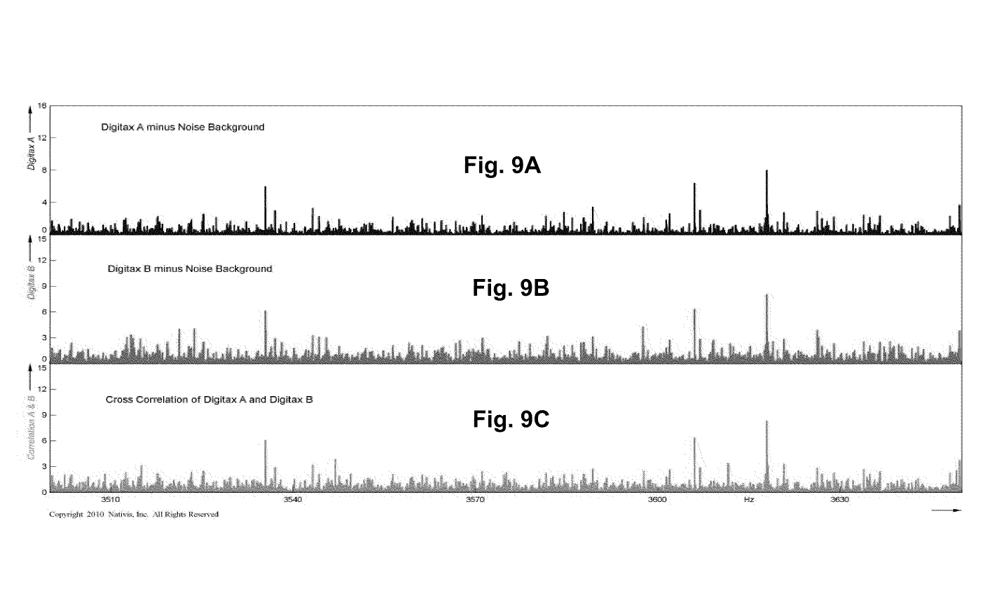

FIGS. 9A-9C show frequency-domain spectra of two paclitaxel signals with noise removed by Fourier subtraction (FIGS. 9A and 96), and a cross-correlation of the two signals (FIG. 9C), showing agent-specific spectral features over a portion of the frequency spectrum from 3510 to 3650 Hz. As can be seen from FIG. 9C, when a noise threshold corresponding to an ordinate value of about 3 is imposed, the paclitaxel signal in this region is characterized by 7 peaks. The spectra shown in FIGS. 9A-9C, but expanded to show spectral features over the entire region between 0-20 kHz, illustrate how optimal time-domain signals can be selected, by examining the frequency spectrum of the signal for unique, agent-specific peaks, and selecting a time-domain signal that contains a number of such peaks.

The time-domain signals recorded, processed, and selected as above may be stored on a compact disc or any other suitable storage media for analog or digital signals and supplied to the transduction system during a signal transduction operation The signal carried on the compact disc is representative, more generally, of a tangible data storage medium having stored thereon, a low-frequency time domain signal effective to produce a magnetic field capable of transducing a chemical or biological system, or in producing an agent-specific aqueous composition in accordance with the invention, when the signal is supplied to electromagnetic transduction coil(s) at a signal current calculated to produce a magnetic field strength in the range between 1 G and 10⁻⁸ G. Although the specific signal tested was derived from a paclitaxel sample, it will be appreciated that any taxane-like compound should generate a signal having the same mechanism of action in transduced form.

One class of time-domain signals produced and selected by the methods above includes signals derived from a taxane- or taxane-like compound, as detailed above for paclitaxel. Another general class of therapeutic compounds contemplated in the present invention are therapeutic oligonucleotides, including single-stranded (ss) and double-stranded (ds) RNA, DNA, and ss and ds oligonucleotide analogs, such as morpholino, phosphorothioate, and phosphonate analogs with various backbone and/or base modifications. These compounds function in a therapeutic role when present in a cellular environment, typically by inhibiting or activating the expression of one or more selected cellular proteins.

### IV. Transduction/Exposure Apparatus and Protocols

This section describes equipment and methodology for exposing and an aqueous medium to low-frequency time-domain signals generated and selected according to the methods described in Sections I-III above, in generating the aqueous composition of the present invention. It will be understood that the term “transducer” or “transducer apparatus” or “transducer/exposure apparatus” or “exposure apparatus,” as employed herein, refers to an apparatus that may function in either a transduction mode, in transducing a biological system that is placed in the magnetic-field environment of the apparatus, or in an exposure mode, for use in producing the aqueous medium of the invention by exposing an aqueous medium in accordance with the invention.

### A. Transducer/Exposure Device and Method

One general type of transducer/exposure device, shown at 500 in FIG. 7, is designed for detecting changes in an optical characteristic of the system in response to transduction, or for detecting changes in an aqueous composition in response to exposure to an impressed time-domain signal. This device includes an optically transparent cell 502, which serves as the transduction/exposure station in the device, and a spectrophotometer, including a electromagnetic beam source 504 and a photodetector 506, for detecting beam absorption and/or emission from the sample. One exemplary sample cell is a 70 μL volume quartz cuvette. Transduction coils 510 located at opposite end regions of the cell were engineered and manufactured by American Magnetics to provide uniform magnetic field strength between coils, and leads for the two coils are shown at 512, 514. In an exemplary embodiment, each coil consists of 50 turns of # 39 gauge (awg) square copper magnet wire, enamel coated, with about a diameter 7.82 mm air core. Suitable spectrometers include (i) a UV or UV-Vis absorption spectrometer, (ii) an IR absorption spectrometer, including one equipped with FTIR capability, or (iii) a Raman spectrometer, all as referenced above.

In another general embodiment of the transducer/exposure device, several Helmholtz coil pairs may be constructed to be orthogonal to one another. This configuration would allow greater flexibility in controlling the structure of the magnetic field applied to a sample. For example, a static magnetic field could be applied along one axis, and a varying magnetic field applied along another axis. The transducer/exposing apparatus described above are placed in a shielded enclosure for the purpose of minimizing uncontrolled extraneous fields from the environment in the region where the sample is placed. In one embodiment of the shielding, the transduction equipment is located within a much larger enclosure, a least 3 times larger than the transduction equipment. This large container is lined with copper mesh attached to Earth ground. Such a container is commonly called a “Faraday cage”. The copper mesh attenuates external environmental electromagnetic signals that are greater than approximately 10 kHz.

In a second embodiment of the shielding, the transduction equipment is located within a large enclosure constructed of sheet aluminum or other solid conductor with minimal structural discontinuities. Such a container attenuates external environmental electromagnetic signals that are greater than approximately 1 kHz.

In a third embodiment of the shielding, the transduction equipment is located within a very large set of three orthogonal Helmholtz coil pairs, at least 5 times larger than the transduction equipment. A fluxgate magnetic sensor container is located near the geometric center of the Helmholtz coil pairs, and somewhat distant from the transduction equipment. Signal from the fluxgate sensor is input to a feedback device, such as a Lindgren, Inc, Magnetic Compensation System, and a feedback current used to drive the Helmholtz coils, forcing a region within the Helmholtz coils to be driven to zero field. Since the Helmholtz coil pairs are very large, this region is also correspondingly large. Furthermore, since the transduction equipment uses relatively small coils, their field does not extend outward sufficiently to interfere with the fluxgate sensor. Such a set of Helmholtz coil pairs attenuates external environmental electromagnetic signals between 0.001 Hz and 1 kHz.

In a fourth embodiment of the shielding, the transduction equipment may be located in either a copper mesh or aluminum enclosure as mentioned above, and that enclosure itself located within the set of Helmholtz coil pairs mentioned above. Such a configuration can attenuate external environmental electromagnetic signals over their combined ranges.

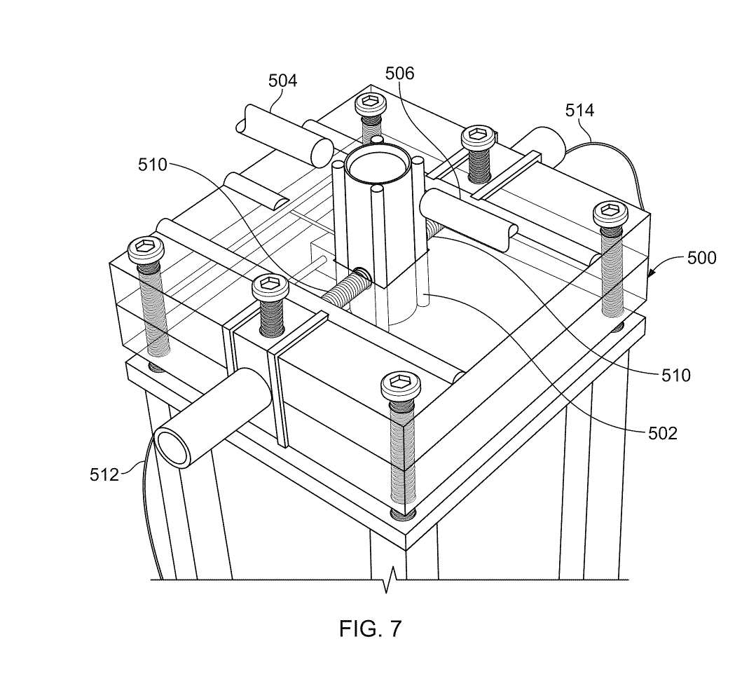

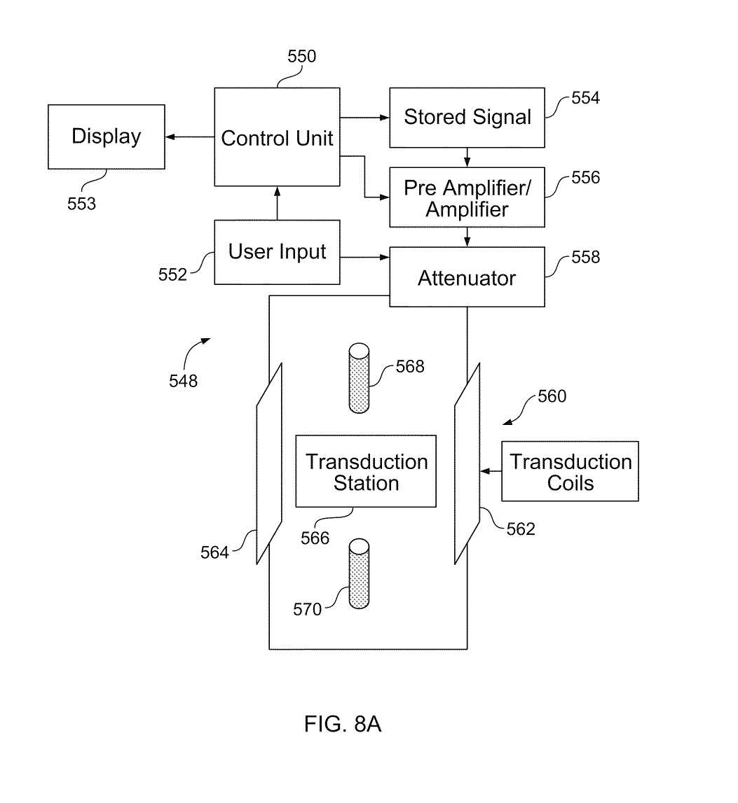

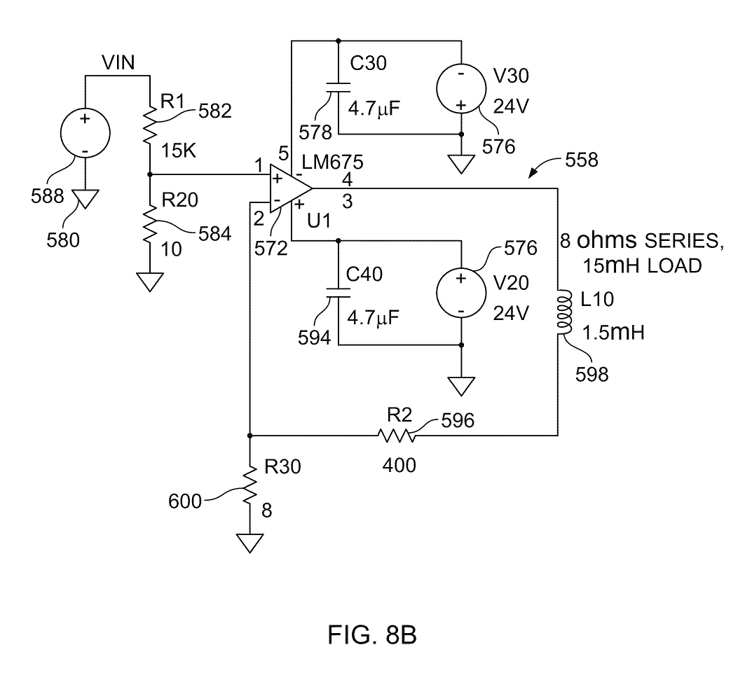

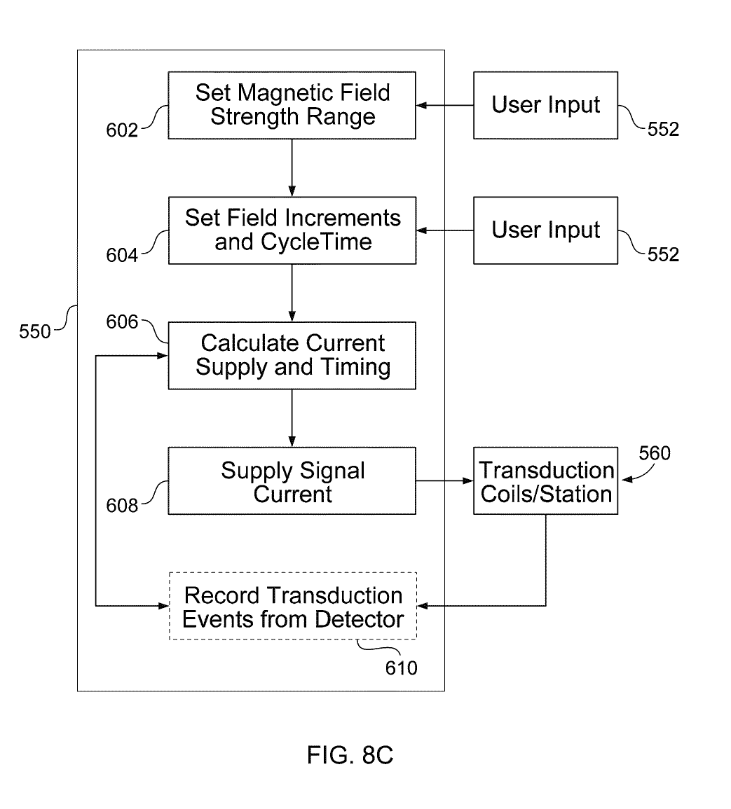

Each of the transducer/exposure devices described above forms part of a system or apparatus that includes components for converting a time-domain signal to a signal-related magnetic field at the transduction/exposure station of the device. FIG. 8A illustrates a general transduction/exposure system 548 having a transducer 560 composed of a pair of transduction coils 562, 564 at opposite ends of a transduction station 566. As indicated above, the transduction station receives either an response system that can respond in a detectable way to an agent-specific signal, or an aqueous medium that is to be exposed in accordance with the invention. The transducer shown in the figure also includes spectrometer components 568, 570.

A control unit 550 in the system is designed to receive user input from an input device 552, and display input information and system status to the user at a display 553. As will be considered in FIG. 8C below, the user input typically includes information specifying the magnetic field strength or range or magnetic field strengths that will be applied during transduction or exposure operations, specifying various timing variables, such as field-increment and field-cycle times, as well as total transduction/exposure time, as will be considered below. Based on this input, the control unit calculates settings that will be applied to the signal-amplifying and attenuating components in the system to achieve the desired transduction or exposure magnetic field strengths over the selected time periods.

A source of stored time-domain signal in the system is indicated at 554. Where the time-domain signal is recorded on a CD or other storage medium, the signal source includes the medium and a medium player, and as seen, is activated by the control unit. Alternatively, where the signal source is transmitted from a remote station via a wireless receiver or Internet connection, the source includes the remote signal source and the receiver or connection. The signal source is connected to a conventional pre-amplifier/amplifier 556 also under the control of unit 552, which outputs an amplified signal voltage to an attenuator 558, also under the control of unit 550. As will be seen below with reference to FIG. 8B, the purpose of the attenuator is to convert signal voltage output from amplifier 556 to a signal current output, and to attenuate the output current to the transducer colts to produce a selected range of magnetic field strengths or a selected magnetic field. The attenuator can be set to produce selected magnetic fields having very low field strengths, e.g., in the range 1⁻⁵ G to 10⁻⁸ G, although the range of producible field strengths may be much greater, e.g., 1 G or 10⁻⁸ G. The control box, amplifier/preamplifier, and attenuator are also referred to herein collectively, as an electronic interlace between the signal source and the electromagnetic coil device.

In one general embodiment, the system is set by the user to supply voltage and current settings to the amplifier, preamplifier and attenuator to achieve incremental magnetic fields from about 1 G to 10⁻⁸ G, over about 50 increments, where the settings for each increment are maintained for 1-5 seconds and the system continuously cycles through the range of field strengths over a user-selected transduction period, e.g., 20 minutes up to several days.

The signal is supplied to the electromagnetic coils 562 and 564 through separate channels, as shown. In one embodiment, a Sony Model CDP CE375 CD Player is used. Channel 1 of the Player is connected to CD input 1 of Adcom Pre Amplifier Model GFP 750. Channel 2 is connected to CD input 2 of Adcom Pre Amplifier Model GFP 750. CD's are recorded to play identical signals from each channel. Alternatively, CD's may be recorded to play different signals from each channel. A Gaussian white noise source can be substituted for signal source 554 for use as a white-noise transduction control. Although not shown here the system may include various probes for monitoring conditions, e.g., temperature within the transduction station.

The circuit diagram for an embodiment of attenuator 558 in FIG. 8A is shown in FIG. 8B, including a power amplifier 572 such as the National Semiconductor LIV1675 Power Operational Amplifier. The power amplifier 572 provides wide bandwidth and low input offset voltage, and is suitable for DC or AC applications, among other benefits. The power amplifier 572 is connected via pin 1 to an input Voltage 588, which is connected to ground 580 either directly or via one or more resistors (582, 584) acting to divide the input, Pin 2 is connected to ground, via a resistor 600. A DC power source 576, such as a regulated and filtered 24 Volt DC power source in parallel with capacitors 578 and 594, is connected to the power amplifier 572 at pin 3 and pin 5. The output of the amplifier (pin 4) is connected to an inductor 598, such as an 8.5 Ohm inductor.

Typical attenuation for such a circuit is approximately 90 dB. However, connecting the inductor 150 to ground 600 via a small resistance, such as the 400 Ohm resistor 596, provides additional attenuation, enabling the system to produce low output currents, as well as other benefits. The system may vary the attenuation by varying the value of the resistor 596, which in turn varies the output current. Additionally, the system may implement a low pass RC filter in series between the inductor 598 and ground 600 to eliminate or minimize self oscillation caused by any self generated tones within the circuit.

More generally, transduction/exposure by an incremented magnetic field produced by a signal current rather than signal voltage, and/or calculated to produce a selected range within 1 G and 10⁻⁸ G, e.g., 10⁻³ to 10⁻⁸ G, or 10⁻⁶ to 10⁻⁸ G, represent an improved transduction/exposure method over earlier methods employing magnetic fields generated by signal voltage and/or at constant magnetic field strength and/or at field strengths greater than about 10⁻⁵ G.

The operational features of the transduction/exposure system 548 in FIG. 8A are illustrated in FIG. 8C, where the control unit, signal source, pre-amp and amp, and attenuator, which collectively make up the electronic interface in the system, are indicated by the dashed line box 550. The transduction system 560 in the figure may be, for example, the coil configuration in FIG. 7, or variants thereof. As seen, the control unit is initially set by user input at 552 to a specified magnetic-field strength or incremented field-strength range desired at the transductions coils (box 602), and also set by the user to desired field increments and cycle times (box 604). For example, the user may specify a constant magnetic field strength, typically between 10 and 10⁻⁸ G, e.g., 10⁻⁵, 10⁻⁶, 10⁻⁷, or 10⁻⁸ G, or an incremented range of magnetic field strengths between 1 G and 10⁻⁸ G, such as a range between 1 G and 10⁻⁸ G or between 10⁻⁵ to 10⁻⁸ G. For a constant field strength, the user may then input desired on and “off” periods and total transduction period, for example, 5 minutes “on” 1 minute “off” over a total transduction period of 1 24 hours. Where an incrementing field-strength range is initially selected, the user will additionally specify the field-strength increments and total increment times, for example, 50 equal increments over 1 G and 10⁻⁸ G, at increment times of 12 second each, for a total cycle time of ten minutes. In the incremented field strength operation, the control unit preferably operates to place a short “off” interval, e.g., one millisecond, between each incremented “on” interval, so that the target is exposed to discrete pulses of incremented magnetic pulses within each cycle.

One preferred transduction coil configuration is composed of two side by side electromagnetic coils on either side of the transduction/exposure station. The magnetic field strength within the coil environment, as a function of the current level of the applied time-domain signal, can be calculated by well-known methods, for example, as indicated at box 606 in the figures, and as detailed on pages 122 to 142 of Applications of Maxwell's Equations, Cochran, J. F. and Heinrich, B., December, 2004. This calculation is done at 606 in the control unit. In one preferred embodiment, the signal current applied to the coils is incremented every 1-5 seconds, in 0.5 to 99.5 dB increments of magnetic field strength, to produce a calculated magnetic field strength that begins at nominal 10⁻⁸ G, and over a range of 1 to 99 steps, achieves a nominal maximum field strength of 1 G, at which point a new cycle of magnetic-field pulses over the same range is begun. The interval between successive equal intensity magnetic-field pulses is preferably in the range of 1-100 sec.

The transduction/exposure parameters, i.e., the selected transduction/exposure conditions to which the system is exposed are (i) the current of the applied time-domain signal, (ii) the duration of applied signal, and (iii) the scheduling of the applied signal. The applied current may be over a range from slightly greater than zero to up to about 1000 mAmps. The total time of transduction may be from a few minutes to up to several days.

The box indicated at 608 in FIG. 8C includes the signal source, pre-amp and amp, and attenuator shown in FIG. 8A. These components are activated and controlled by the control unit to supply the desired current, current increments, cycle and total transduction times stored in the unit. The current output from the attenuator is delivered to the transduction coil(s) 560, as indicated, to produce the desired magnetic-filed strength in the transduction/exposure station. Where the course of transduction events can be monitored by a change in the optical (or other measurable) change in the target system, this information is fed to a component 610 in the control unit, and this information may be used to control transduction conditions, by feedback to component 606, and/or displayed to the user for purposes of manually controlling transduction/exposure conditions.

### V. Preparation and Agent-Specific Activity of Aqueous Pharmaceutical Compositions Generated from Paclitaxel Signals

In one aspect, the invention includes an aqueous anti-tumor composition produced by treating an aqueous medium free of paclitaxel, a paclitaxel analog, or other cancer-cell inhibitory compound with a low-frequency, time-domain signal derived from paclitaxel or an analog thereof, until the aqueous medium acquires a detectable paclitaxel activity. The agent-specific activity is evidenced by the ability of the composition (i) to inhibit growth of U87 MG human glioblastoma cells when the composition is added to the U87 cells in culture, over a 24 hour culture period, under standard culture conditions, and/or (ii), to inhibit growth of a paclitaxel-responsive tumor when administered to a subject having such a tumor. This section describes the preparation of exemplary compositions, one in which the aqueous medium is a cell-culture medium, and the other in which the aqueous medium is ultrapure water, and the agent specific activity of the compositions.

### A. Preparation of Paclitaxel-Signal Compositions

DME medium (Invitrogen SKU# 10313-021 (Carlsbad, Calif.) medium supplemented with 4,500 mgs/l D-glucose) was placed in 35 ml glass vials and equilibrated to room temperature. The medium was vortexed for 20 seconds at the maximum setting of a Vortex mixer (VWR, Westchester, Pa.), and placed at the sample station of a transduction/exposure apparatus having a solenoid coil for field generation.

The medium-containing vial as exposed to the taxane M2(3) signal described above for 20 minutes, at current levels calculated to produce magnetic field strengths in one of three selected ranges: 1 G to 10⁻¹ G (Range 1); 10⁻² G to 10⁻³ G (Range 2), and 10⁻⁵ G to 10⁻⁶ G (Range 3), where for each selected range, the signal was alternated between the two field-strength extremes, top to bottom, then back to top, in 0.5 dB increments played for 1 sec each. Thus, for example in Range 1, the initial attenuator setting was calculated to produce a magnetic field strength of 1 G, and then decremented in 0.5 dB steps, 1 sec/step until the lower 10⁻¹ G range was reached, at which point the cycle repeated, carried out over a 20 minute period.

At the end of the 20 minute exposure period, the vial was removed from the coil station and vortexed again for 20 seconds under the same pre-exposure vortexing conditions. The exposed medium was used immediately in the cell-culture medium studies detailed below.

A taxane-signal water medium was prepared by identical methods, substituting ultrapure water (double-distilled) for the cell culture medium, and including the 20-second vortexing steps before and after exposure to the taxane signal for 20 minutes, within each of the three ranges specified above.

### B. Inhibition of Human Glioblastoma Cells Grown in a Paclitaxel-Signal Cell-Culture Composition

U87 MG human glioblastoma cells were purchased from American Type Culture Collection (ATCC, Rockville, Md., USA), The cells were grown in complete DMEM growth medium (invitrogen) supplemented with 4,500 mg/l D-glucose plus Pen/Strepp/Glu and non-essential amino acids The cells were seeded in cell culture flasks (75 ml) and incubated at 37° C. in a fully-humidified atmosphere with 5% CO₂. Once the cells reach confluence, they were propagated and/or preserved as described below:

For propagation, the medium was removed and the attached cells were washed 2× with PBS, then treated with trypsin until the cells detached. Fresh medium was added, and the cell suspension was dispensed in new culture flasks. For preservation, the cells were frozen in 95% complete growth medium supplemented by 5% DMSO.

For signal transduction, 2,500 U-87 cells in about 100 μl were added to each of 6 wells in a 96 well microtitre plates and allowed to settle overnight at 37° C. The medium from the wells was removed and replaced with 100 μl of fresh medium (6 wells), or fresh medium exposed to a taxane signal at a selected magnetic field setting. The plates were then incubated at 37° C. in a fully-humidified atmosphere with 5% CO2.

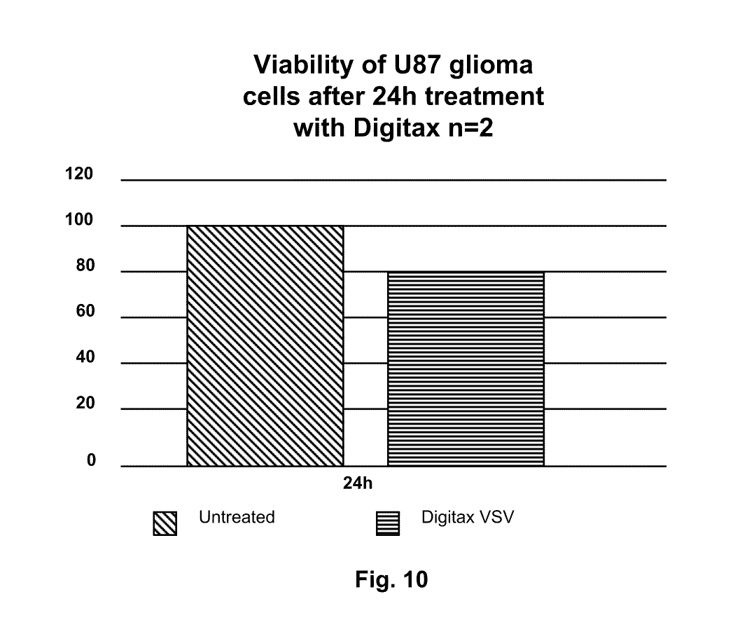

Twenty-four hours after addition of fresh medium, 10 μl of AlamarBlue viability dye was added to 6 wefts in each of the five groups, and the total fluorescence, as a measure of viable cell count, was measured. The cell count was converted to a percentage cells relative to the average cell number in the untreated wells, as a measure of cell-growth inhibition. The results of the study, given in FIG. 10, show that after 24 hours, the cells growing in taxane-signal exposed medium were experiencing a significant cell-growth inhibition effect, in the range of about 20% inhibition,

### C. Treatment of Glioblastoma Tumors in Mice a Paclitaxel-Signal Ultra-Pure Water Composition

The ability of water exposed to a taxane signal to inhibit a glioblastoma tumor in an animal model was investigated. In this study, nine groups of 8 mice were each injected in the right flank with 7.355×10⁶ U-87 glioblastoma cells, and treatment with the various modalities was begun either one day after inoculating the animals with the cells, or when the tumors reached 75-100 mm³. The treatment groups are given in Table 1 below:

Paclitaxel was initially dissolved in a 1:1 v/v mixture of CremaphorEL and ethanol and stored at 4° C. Final dilution of the drug to a concentration of 1.5 mg/ml was made with 0.9% NaCl immediately before use. In Groups 3 and 4, the paclitaxel was administered by intravenous injection into the tail vein at a dose of 15/mg/kg animal weight on each of five consecutive days. The actual volume administered to each animal was about 0.2 ml of the above paclitaxel formulation.

Animals in Groups 5 and 6 received 20 ml/kg (or about 0.5 ml) of water exposed to taxane signal prepared as described in Section VII, with the Range 1 magnetic field. The exposed water composition was administered by oral gavage immediately after preparation, either a day after inoculating the animals with the tumor cells (Group 5) or when the tumor volume reached 75-100 mm³ (Group 6). Animals in Group 7 had no tumors, but were treated with the M2(3) water, Animals in group 8 and 9 were treated identically to those in Groups 5 and 6, respectively, except that the exposed water administered to the animals was prepared by exposing ultrapure water to a white noise signal, Administration was once daily throughout the remainder of the study until day 26.

For all groups, tumor volumes were measured every other day by serial caliper measurements to determine tumor width and length, and calculating an approximate tumor volume from the formula: Tumor volume=(length×width²)/2.

TABLE 1
Treatment Groups

| | Tumor Cell | | Route of | |
|---|---|---|---|---|
| Group # | Injection site | Treatment | Administration | Start of Treatment |
| 1 | Right flank | None | NA | NA |
| 2 | Right flank | Paclitaxel Vehicle | IV | Day after cell |
| | | | | inoculation |
| 3 | Right flank | Paclitaxel 15 mg/kg | IV | Day after cell |
| | | qdx5 | | inoculation |
| 4 | Right flank | Paclitaxel 15 mg/kg | IV | After tumors reach |
| | | qdx5 | | predetermined size |
| 5 | Right flank | Water exposed to | Oral gavage | Day after cell |
| | | M2(3) 0.5 ml/animal, | | inoculation |
| | | once daily until | | |
| | | sacrifice | | |
| 6 | Right flank | Water exposed to | Oral gavage | After tumors reach |
| | | M2(3), 0.5 ml/animal, | | predetermined size |
| | | once daily until | | |
| | | sacrifice | | |
| 7 | No tumor | Water exposed to | Oral gavage | Day after cell |
| | cells | M2(3), 0.5 ml/animal, | | inoculation |
| | | once daily until | | |
| | | sacrifice | | |
| 8 | Right flank | Water exposed to | Oral gavage | Day after cell |
| | | white noise, 0.5 | | inoculation |
| | | ml/animal, once daily | | |
| | | until sacrifice | | |
| 9 | Right flank | Water exposed to | Oral gavage | After tumors reach |
| | | white noise 0.5 | | predetermined size |
| | | ml/animal, once daily | | |
| | | until sacrifice | | |

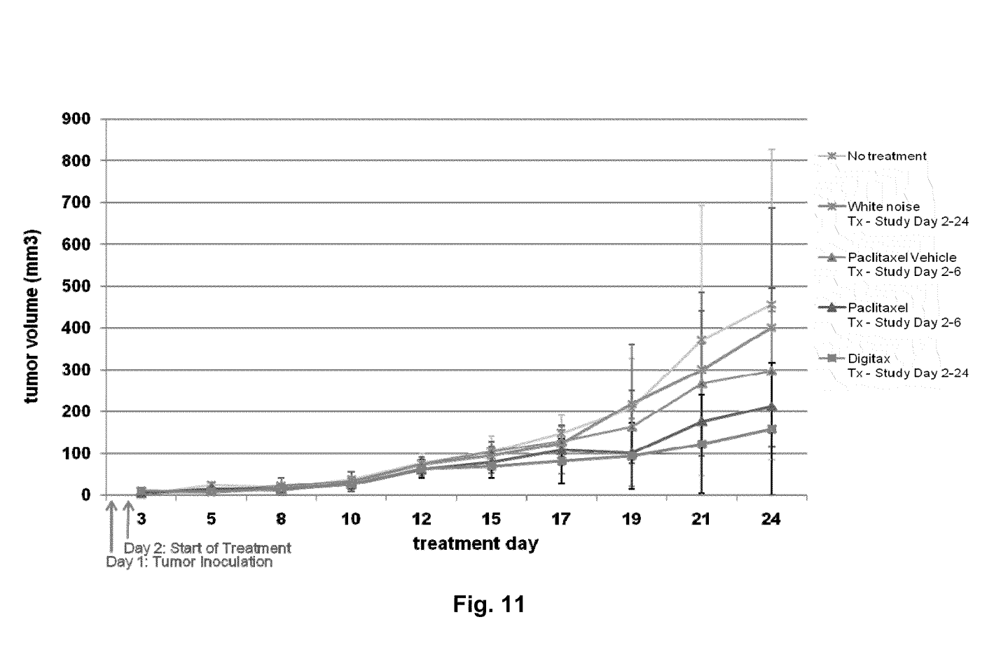

Considering now the results of the study, FIG. 11 is a plot of tumor volumes from the untreated animal group (Group 1, X's, light line); animals treated with white noise water (Groups 8 and 9, X's heavy line); animals treated with paclitaxel vehicle (Group 2, triangles, light line), animals treated with paclitaxel (Groups 3 and 4, triangles, heavy line); and animals treated with taxane-water (Groups 5 and 6, squares, heavy line), over a 24-day period. Similar to the results observed for paclitaxel, the increase in tumor volume over the study period was more than twice as high in untreated (about 650 mm³) than in animals treated with the water exposed to a taxane signal.

These studies, though preliminary, are consistent with the cell culture studies presented above in demonstrating that an aqueous medium, including both cell-culture medium and ultrapure water, can be influenced with an agent-specific signal, such that the medium itself can produce effects that mimic the signal even after the signal is removed.

### VI. Therapeutic Signals and Compositions Derived from Oligonucleotides

In another aspect, the invention includes an aqueous composition produced by treating an aqueous medium free of oligonucleotide with a low-frequency, time-domain signal derived from a therapeutic oligonucleotide, until the aqueous medium acquires a detectable activity associated with the therapeutic oligonucleotide. Exemplary therapeutic oligonucleotide from which the signals and compositions are derived include GAPDH antisense RNA or PCSK9 antisense RNA. Methods for generating oligonucleotide-specific signals and aqueous compositions and a demonstration of the agent-specific activity of the four different compositions is given in subsections A and B below.

### A. GAPDH Antisense RNA

Tumor cells characteristically exhibit an increased rate of glycolysis, and in some cancers, this increase is attributable to a higher level of glyceraldehyde-3-phosphate dehydrogenase (GAPDH). It has been reported, for example, that levels of GAPDH gene expression are strongly elevated in three cervical carcinoma cell lines (HeLa, CUMC-3, and CUMC-6) compared with normal cervical tissue. (Kim, J. W., et al., Antisense Nucleic Acid Drug Dev. 1999 December; 9(6):507-13). The same study showed that GAPDH antisense resulted in reduced cellular proliferation, which was accompanied by reduced colony-forming efficiency. This effect of GAPDH antisense on cultured carcinoma cells was associated with the apoptotic process, including increased DNA fragmentation.

Preparation of GAPDH antisense RNA signals and compositions: A GADPH antisense RNA molecule having the sequence identified by SEQ NO: 1 was dissolved in water to a final concentration of 10⁻¹⁵ μM, and the solution was vortexed for 20 seconds immediately before signal recording. Signal recordings were performed as described in Section UI above. A control GAPDH antisense RNA with a non-targeting sequence (SEQ ID NO: 2) was similarly prepared and its signal recorded. A high-scoring time-domain signal was used to treat culture medium, as described in Section VIIA above in the paclitaxel studies,

Methods and results. After 48 hours in culture, cells growing in the antisense signal-treated culture medium showed 78% GAPDH activity relative to 100% level for control cells grown in culture medium formed by treating culture medium with the non-targeting sequence signal.

### B. PCSK9 Antisense RNA

Loss-of-function mutations of proprotein convertase subtilisin/kexin type 9 (PCSK9) have been shown to increase the density of the LDL-R on the hepatocyte cell membrane and increase the rate of removal of LDL from plasma and lower LDL levels. Thus, it is expected that strategies that result in the inhibition of PCSK9 synthesis or inhibition of the binding of PCSK9 to the LDL-R should lower plasma cholesterol levels, and this effect has been demonstrated with antisense oligonucleotides against PCSK9 and polyclonal antibodies against PCSK9

Preparation of PCSK9 antisense RNA signals and compositions. A PCSK9 antisense RNA molecule having the sequence identified by SEQ NO: 3 was dissolved in water to a final concentration of 10⁻¹⁵ μM, and the solution was vortexed for 20 seconds immediately before signal recording. A control, non-targeting sequence has SEQ ID NO: 2 above. Signal recordings were performed as described in Section III above. A high-scoring time-domain signal was used to prepare a signal-water composition.

Methods and results, C57BL/6J mice, 12-13 weeks old, were divided into two groups of 5 animals each: a control group that received double-distilled water and a treatment group that received signal-activated water. Dosing was by oral gavage, 0.5 ml at time 0 and 12 hours. At 24 hours, the animals were sacrificed, blood was removed for lipid-chemistry workup and livers were removed to assay for liver pCSK9 mRNA, according to standard methods.

As seen in FIG. 12A, LDLc levels, expressed relative to control (100%), declined to 64% at day 1, where the individual values for the five control animals and five treated animals are shown in FIG. 12B. HDLc levels remained substantially constant after one day, as seen in FIG. 12C. Triglyceride levels showed an overall decline, as seen for the individual control and treated animals in FIG. 12D. A dramatic knockdown of pCSK9 mRNA (about 90%) was observed in several animals, whereas others showed little knockdown effect.

### VII. System for Producing and Confirming the Activity of a Pharmaceutical Composition

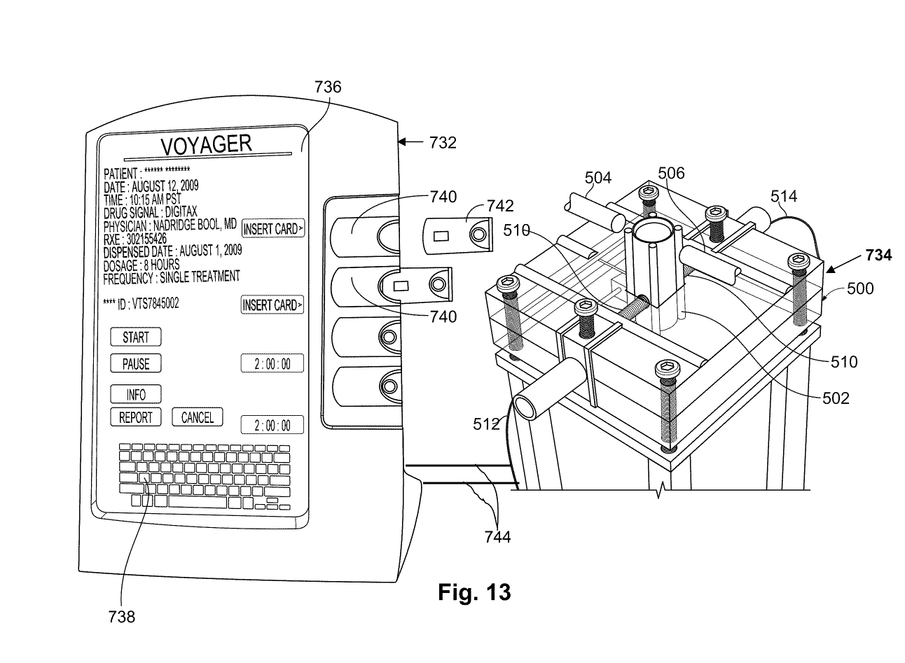

Also forming a part of the invention is a system for producing an aqueous composition intended to produce an agent-specific pharmaceutical effect on a mammalian subject, when the composition is administered in a pharmaceutically effective amount to the subject, and for An exemplary system is shown at 730 in FIG. 13, and includes an electronic unit 732 for outputting a drug-derived time-domain signal at a selected current level, and an activation unit 734 for activating an aqueous medium to produce the drug-signal composition of the invention, and for testing the activity of the composition.

Unit 732, referred to as a Voyager™ unit, includes substantially the same components as control unit 550 described above with respect to FIGS. 8A-8C, including a display 736, keys for user input 738, and circuitry and software for converting a low-frequency time domain signal into a signal output having a current level calculated to produce a magnetic field of a selected field strength at the activation unit. Also included in the unit is a plurality of card readers, such as readers 740, each for receiving a drug-signal card 742 having a suitable storage medium on which is stored a selected-drug low-frequency time domain signal produced and selected as detailed above. For example, one of cards 740 may include a paclitaxel time-domain signal such as employed in the above in vitro and in vivo studies. In another general embodiment, drug-signal cards and card reader are replaced by signal-storing CD-ROMs and one or more internal CD-ROM players, or by a suitable transceiver for receiving a requested drug-derived low-frequency time-domain signal, e.g., via phone or Internet line.

The output of the Voyager control unit is connected to the activation unit 734 through shielded wires 744, which connect to unit to conductors 512, 514 connected the conductive-wire coils 510 used in generating the desired magnetic field within the interior of a activation station 502 in the unit. The station is dimensioned to receive a container or vial containing an aqueous medium, e.g., ultrapure water or liposome suspension that is to be activated to the drug-signal composition of the invention. The coil windings are similar in those described above with respect to FIG. 7 or may be a single solenoid coil within which the sample is held. The coil(s) are designed to produce a uniform magnetic field within the activation station. The system may additionally contain a tabletop vortexing device for agitating the drug-signal contents before and/or after exposure to the drug signal.

Using this system, a pharmacist or physician can readily generate raw drug-signal compositions on request and in a time of no more than about 10-30 minutes/per sample. For larger scale needs, e.g., multiple patient treatment, the system may include multiple exposure stations at which single-dosage compositions may be produced in batch form, or may be scaled up to generate composition volumes suitable for multiple doses.

Once a composition is produced, its drug-equivalent activity may be confirmed by spectroscopic means, such as by ultraviolet spectroscopy, Fourier-transform (FT) infrared spectroscopy and/or Raman spectroscopy, all of which are capable of detecting spectral features associated with structured water (Rao, M. L., et al., Current Science, 98(11):1500 Jun., 2010). In this approach, the UV, infrared, and/or Raman spectra of each of a series of signal compositions having different known activities are generated in advance, to serve as standards against which an unknown sample spectrum can be compared. Alternatively, the device may include an an atomic force microscope (AFM) capability for detecting changes in water structure. The spectrometer is represented schematically in the figure by a light source 504 and photodetector 506.

UV-Vis (visible) spectroscopy may be carried out with a UV spectrometer and according to methods described, for example, by Chai, B., et al., J. Phys Chem A. 2008, 112:2242-2247. As described there, absorption-spectral measurements are performed on a single beam Hewlett-Packard (Model 8452A) diode-array spectrophotometer. A UV quartz micro-rectangular cuvette (Sigma Aldrich) is used, with inside dimensions 12.5 mm length, 2 mm width, and 45 mm height. The transmitting range of the cuvette is from 170 nm to 2.71 m, The light-path length in the cuvette is 2 mm. The displayed spectra are averages of at least ten scans.

IR spectroscopy may be carried out by conventional means, as described for example, in Roy, R., Materials Res. Innov, 2005, 9(4):1433 and Rao, M., et al., Materials Letters, 2008, 62(10-11):1487-1490). The IR spectrometer may be equipped for performing Fourier-transform infrared absorption (FTIR) spectroscopy, as described, for example, by Amrein, A., et al., J. Phys Chem, 1988 92(19): 5455-5466). Raman spectroscopy is carried out using well-known Raman spectroscopy tools, where separate Raman spectra may be taken, for example, at 785 nm and 532 nm.

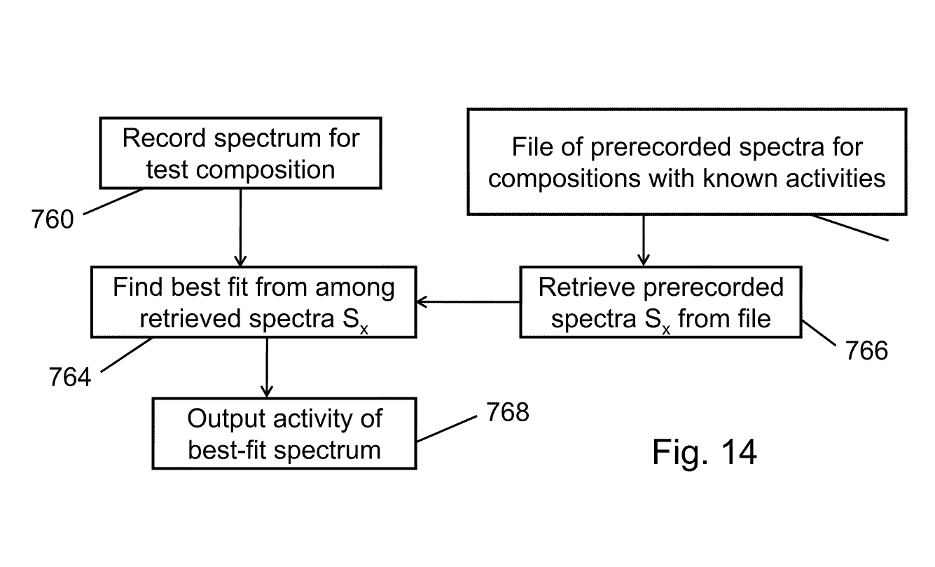

FIG. 14 is a flow diagram of steps carried out in the system for determining or confirming the agent-specific activity of an aqueous composition formed in the system. As indicated at 762 in the figure, the system includes a file of spectra, e.g., UV, UV-V is, IR, or Raman, spectra that have been prerecorded for aqueous compositions with known agent-specific activities. Thus, for example, the file may include a number of spectra taken for aqueous compositions formed under different exposure conditions to a paclitaxel signal, and tested for paclitaxel activity, e.g., in a cell culture system. Thus, each spectrum corresponds to a given, tested activity.

After recording the spectrum of a test sample newly formed in the system, each of the Sₓ prerecorded spectra are successively retrieved, at 766, and matched against the test spectrum, at 764. This matching may be carried out by a conventional curve-matching method, such as by generating a difference spectrum, and quantitating one of more features of the difference spectrum, such as the ratio of peak heights at selected frequencies. Once an optimal match to a prerecorded spectrum Sₓ is identified, at 764, the activity corresponding to the best-fit spectrum is displayed to the user, to determine or confirm an activity for the signal-impressed composition.

More generally, the invention includes a method confirming the agent-specific activity of the signal composition of the invention, by (a) measuring the spectrum of the composition by one or (i) ultraviolet spectroscopy, (ii) infrared spectroscopy, and (iii) Raman spectroscopy, and (b) determining that the measured spectrum is similar in its spectral composition and amplitudes to a spectrum having a known cancer-cell inhibitory activity.

The invention further provides a system for producing an aqueous composition intended to produce an agent-specific pharmaceutical effect on a mammalian subject, when the composition is administered in a pharmaceutically effective amount to the subject. The system includes (a) device for treating an aqueous medium with an agent-specific signal under conditions effective to convert the aqueous medium to an aqueous composition having agent-specific properties; and (b) a spectroscopic instrument for generating a spectrum of the composition by one or (i) ultraviolet spectroscopy, (ii) Fourier-transform infrared spectroscopy, and (iii)

Raman spectroscopy, thus permitting confirmation that the measured spectrum is 9 similar in its spectral composition and amplitudes to a spectrum having a known agent-specific effect.

The system may further include a device for treating the aqueous medium to produce one of; (i) a mechanically disrupted aqueous medium, (ii) an interfacial aqueous medium containing gas bubbles and (iii) a mechanically disrupted interfacial aqueous medium containing gas bubbles. For example, the device may be a vortexing device for mechanically disrupting the composition.

While specific embodiments of, and examples for, the invention are described above for illustrative purposes, various equivalent modifications are possible within the scope of the invention, as those skilled in the relevant art will recognize. In particular, it will be recognized that methods of producing signal-specific effects in a chemical, biochemical, or biological system, by exposing the system to an agent-specific time-domain signal, in accordance with the transduction methods described herein, may be acting directly on the target components of the system or may be acting through a mechanism in which the aqueous medium of the target system is being altered to produce signal-specific effects, even after the signal is turned off, or a combination of the two mechanisms, An important implication of the altered-state mechanism is that relatively brief periods of exposure of a subject to a transducing signal may able be effective to produce extended drug effects, e.g., over a 1-24 hour period.

The teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

### Sequence Listing

SEQ ID NO 1: Antisense strand targeting GAPDH

5′- AAA GUU GUC AUG GAU GAC CTT -3′

SEQ ID NO 2: Antisense strand of non-targeting control

5′- GGG UUG CGC UUA CUU ACG ATT -3′

SEQ ID NO 3: Antisense strand of targeting PCSK9

5′- UCC GAA UAA ACU CCA GGC CTA -3′

It is claimed:

1. An aqueous anti-tumor composition produced by treating an aqueous medium free of paclitaxel, a paclitaxel analog, or other cancer-cell inhibitory compound with a low-frequency, time-domain signal derived from paclitaxel or an analog thereof, until the aqueous medium acquires a detectable paclitaxel activity, as evidenced by the ability of the composition (i) to inhibit growth of human glioblastoma cells when the composition is added to the cells in culture, over a 24 hour culture period, under standard culture conditions, and/or (ii), to inhibit growth of a paclitaxel-responsive tumor when administered to a subject having such a tumor.

2. The composition of claim 1, wherein the aqueous medium is a mechanically disrupted aqueous medium, an interfacial aqueous medium containing gas bubbles, or a mechanically disrupted, interfacial aqueous medium containing gas bubbles.

3. The composition of claim 1, having a activity, expressed in terms of paclitaxel concentration, of between 1 to 100 μM.

4. The composition of claim 1, wherein the aqueous medium includes a suspension of liposomes or other nanoparticles.

5. The composition of claim 1, which includes between 0.05 and 5% ethanol.

6. A method of forming the composition of claim 1, comprising:
(a) placing an aqueous medium within the sample region of an electromagnetic coil device and
(b) exposing the aqueous medium to a magnetic field generated by supplying to the device, a low-frequency, time domain signal derived from paclitaxel or an analog thereof, at a signal current calculated to produce a magnetic field strength in the range between 1 G (Gauss) and 10⁻⁸ G, for a period sufficient to render the aqueous medium effective in inhibiting the growth tumor cells in culture, or inhibiting tumor growth in vivo.

7. The method of claim 6, wherein the low-frequency, time domain signal used in step (b) is produced by the steps of:
(i) placing in a sample container having both magnetic and electromagnetic shielding, an aqueous sample of paclitaxel or analog thereof, wherein the sample acts as a signal source for low-frequency molecular signals; and wherein the magnetic shielding is external to a cryogenic container;
(ii) recording one or more time-domain signals composed of sample source radiation in the cryogenic container, and
(iii) identifying from among the signals recorded in step (ii), a signal effective to mimic the effect of paclitaxel in a paclitaxel-responsive system, when the system is exposed to a magnetic field produced by supplying the signal to electromagnetic transducer coil(s) at a signal current calculated to produce a magnetic field strength in the range between 1 G to 10⁻⁸ G.

8. The method of claim 7, wherein the concentration of the paclitaxel or analog thereof in the sample is between 10⁻¹¹ to 10⁻¹⁹ M.

9. The method of claim 7, wherein the sample is treated, prior to being placed within the sample region of the device, to form one of: (i) a mechanically disrupted sample medium, (ii) an interfacial sample medium containing gas bubbles, (iii) a mechanically disrupted interfacial sample medium containing gas bubbles, and (iv) a suspension of liposomes or other nanoparticles.

10. The method of claim 7, wherein the paclitaxel-specific time-domain signal used in step (b) is produced, in step (iii) of identifying a signal from step (ii) that is effective in promoting the extent of tubulin polymerization in a tubulin suspension, by enhancing polymer formation and/or stabilizing formed polymer, when a suspension of tubulin molecules is exposed to a magnetic field produced by supplying the signal to electromagnetic transducer coil(s) at a signal current calculated to produce a magnetic field strength in the range between 1 G to 10⁻⁸ G.

11. The method of claim 6, further comprising, before and/or after step (b), treating the aqueous medium to form one of: (i) a mechanically disrupted aqueous medium, (ii) an interfacial aqueous medium containing gas bubbles, (iii) a mechanically disrupted interfacial aqueous medium containing gas bubbles, and (iv) a suspension of liposomes or other nanoparticles.

12. The method of claim 11, further comprising, before and/or after step (b) mechanically agitating the aqueous medium by vortexing to form a mechanically disrupted aqueous medium.

13. A method confirming the cancer-cell inhibitory activity of the composition of claim 1 by the steps of:
(a) generating a spectrum of the composition by one or (i) ultraviolet spectroscopy, (ii) Fourier-transform infrared spectroscopy, and (iii) Raman spectroscopy, and
(b) determining that the generated spectrum is similar in its spectral composition to the spectrum of a similarly-prepared aqueous composition having a known cancer-cell inhibitory activity.

14. A method of forming an aqueous composition effective to produce an agent-specific effect on an agent-responsive chemical or biological system, when the composition is added to the system, comprising:
(a) placing an aqueous medium within the sample region of an electromagnetic-coil device;
(b) exposing the aqueous medium to a magnetic field generated by supplying to the device, a low-frequency, time-domain agent-specific signal, at a signal current calculated to produce a magnetic field strength in the range between 1 G (Gauss) and 10⁻⁸ G, for a period sufficient to render the aqueous medium effective in inhibiting the growth of tumor cells in culture, or inhibiting tumor growth in vivo.

15. The method of claim 14, wherein the low-frequency, time domain signal used in step (b) is produced by the steps of: (i) placing in a sample container having both magnetic and electromagnetic shielding, an aqueous sample of the agent, wherein the sample acts as a signal source for low-frequency molecular signals; and wherein the magnetic shielding is external to a cryogenic container;)
(ii) recording one or more time-domain signals composed of sample source radiation in the cryogenic container, and
(iii) identifying from among the signals recorded in step (ii), a signal effective to mimic the effect of the agent in an agent-responsive system, when the system is exposed to a magnetic field produced by supplying the signal to electromagnetic transducer coil(s) at a signal current calculated to produce a magnetic field strength in the range between 1 G to 10⁻⁸ G.

16. The method of claim 15, wherein the concentration of the agent in the sample is between 10⁻¹⁰ to 10⁻¹⁶ μM.

17. The method of claim 14, wherein the sample is treated, prior to being placed within the sample region of the device, to form (i) a mechanically disrupted aqueous medium, (ii) an interfacial aqueous medium containing gas bubbles, (iii) a mechanically disrupted interfacial aqueous medium containing gas bubbles, and (iv) a suspension of liposomes or other nanoparticles.

18. The method of claim 14, further comprising, before and/or after step (b), treating the aqueous medium to form one of: (i) a mechanically disrupted aqueous medium, (ii) an interfacial aqueous medium containing gas bubbles, (iii) a mechanically disrupted interfacial aqueous medium containing gas bubbles, and (iv) a suspension of liposomes or other nanoparticles.

19. The method of claim 18, further comprising, before and/or after step (b) mechanically agitating the aqueous medium by vortexing to form a mechanically disrupted aqueous medium.

20. The method of claim 18, wherein the aqueous medium includes a suspension of liposomes.

21. The method of claim 14, wherein the agent is selected from the group consisting or (i) paclitaxel, (ii) an analog of paclitaxel, and (iii) therapeutic oligonucleotide.

22. The method of claim 21, wherein the therapeutic oligonucleotide is selected from the group consisting of GAPDH antisense RNA and PCSK9 antisense RNA.

23. An aqueous composition produced by treating an aqueous medium free of oligonucleotide with a low-frequency, time-domain signal derived from a therapeutic oligonucleotide, until the aqueous medium acquires a statistically significant activity associated with the therapeutic oligonucleotide.

24. The composition of claim 23, wherein the therapeutic oligonucleotide is selected from the group consisting of GAPDH antisense RNA and PCSK9 antisense RNA.

25. The composition of claim 23, wherein the aqueous medium is a mechanically disrupted aqueous medium, an interfacial aqueous medium containing gas bubbles, or a mechanically disrupted, interfacial aqueous medium containing gas bubbles.

26. The composition of claim 23, wherein the aqueous medium contains between 0.5 to 10% ethanol by volume.

27. A method of confirming the agent-specific activity of the composition of claim 23 by the steps of:
(a) generating a spectrum of the composition by one or (i) ultraviolet spectroscopy, (ii) infrared spectroscopy, and (iii) Raman spectroscopy, and
(b) determining that the generated spectrum is similar in its spectral composition to the spectrum of a similarly prepared aqueous composition having a known agent-specific effect.

28. A system for producing an aqueous composition intended to produce an agent-specific pharmaceutical effect on a mammalian subject, when the composition is administered in a pharmaceutically effective amount to the subject, said system comprising
(a) device for treating an aqueous medium with an agent-specific signal under conditions effective to convert the aqueous medium to an aqueous composition having agent-specific properties; and
(b) a spectroscopic instrument for generating a spectrum of the composition by one or (i) ultraviolet spectroscopy, (ii) Fourier-transform infrared spectroscopy, and (iii)
Raman spectroscopy, thus permitting confirmation that the measured spectrum is similar in its spectral composition and amplitudes to a spectrum having a known agent-specific effect.

29. The system of claim 28, wherein device (a) includes
(a) a source of an agent-specific time-domain signal;
(b) an electromagnetic transduction coil device for receiving a vessel containing an aqueous medium within a vessel holder in the device, and
(c) an electronic interface between said source and said device, for supplying to the device, a source-signal current calculated to produce at an aqueous medium contained in a vessel at the sample region of the device, a magnetic field having a field strength in the range between 1 G to 10⁻⁸ G, over a time period sufficient to transform aqueous medium in said into said agent-specific composition.

30. The system of claim 28, which further includes a device for treating the aqueous medium to produce one of: (i) a mechanically disrupted aqueous medium, (ii) an interfacial aqueous medium containing gas bubbles and (iii) a mechanically disrupted interfacial aqueous medium containing gas bubbles.

31. The system of claim 30, wherein the device for forming a mechanically disrupted aqueous medium is a vortexing device.


SEQUENCE LISTING (3 sequences)

SEQ ID NO: 1 - DNA, 21 bp, Artificial
  synthetic antisense strand targeting GAPDH
aaaguugucauggaugacctt

SEQ ID NO: 2 - DNA, 21 bp, Artificial
  synthetic antisense strand of non-targeting control
ggguugcgcuuacuuacgatt

SEQ ID NO: 3 - DNA, 21 bp, Artificial
  synthetic antisense strand targeting PCSK9
uccgaauaaacuccaggccta